(19) United States
(12) Patent Application Publication
(10) Pub. No.: US 2003/0135880 A1
(43) Pub. Date: Jul. 17, 2003

(54) Compositions and methods for halogenation reactions

(76) Inventors: John Steffens, Chapel Hill, NC (US); Chris Batie, Durham, NC (US); Jon Marquiz Dietz, Apex, NC (US); Jian Dong, Johnston, IA (US); Kim Puloma Kamdar, La Jolla, CA (US); Dwight Steven Hill, Cary, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/148,907

(22) PCT Filed: Dec. 7, 2000
(86) PCT No.: PCT/EP00/12347

(51) Int. Cl. A01H 5/00; C12P 1/00; C12P 21/02; C12N 5/04; C12N 15/82; C12N 9/02; C07D 295/04; C07D 207/04

(52) U.S. Cl. 800/279; 435/41; 435/69.1; 435/189; 435/320.1; 435/419; 548/577

(57) ABSTRACT

This invention describes methods, transgenic plants and transgenic microorganisms for the biosynthesis of halogenated natural products, where the halogenation is substrate and regiospecific. In particular, this invention relates to the use of halogenated metabolites, produced by the method of the invention, for the protection of host organisms against pathogens, more particularly, to the protection of plants against phytopathogens. In this aspect, the invention provides transgenic plants with enhanced resistance to phytopathogens, and biocontrol organisms with enhanced biocontrol properties.

The present invention relates generally to methods, transgenic plants and transgenic microorganisms for the biosynthesis of halogenated natural products, where the halogenation is substrate and regiospecific. In one aspect, the present invention relates to the use of halogenated metabolites, produced by the method of the invention, for the protection of host organisms against pathogens, more particularly, to the protection of plants against phytopathogens. In this aspect, the invention provides transgenic plants with enhanced resistance to phytopathogens, and biocontrol organisms with enhanced biocontrol properties.

Biosynthesis of the over 2000 known naturally-occurring halogenated metabolites has long been regarded as a function of two classes of enzymes: the haloperoxidases and the non-heme haloperoxidases (Gribble G W 1994, The natural production of chlorinated compounds. Environ Sci technol 28:310-319; van Pee K -H [1996] Biosynthesis of halogenated metabolites by bacteria. Annu Rev Microbiol 50:375-99). Of the first group, the bromoperoxidases and chloroperoxidases all possess protoporphyrin IX as the heme-containing prosthetic group. This group acts catalytically by reacting with hydrogen peroxide to form the hydroperoxide of the enzyme (compound I), which then reacts with the halide (X; X═Br⁻, Cl⁻, or I⁻) resulting in the formation of the enzyme (E)-bound intermediate EOX. It is unknown whether EOX is the halogenating agent or whether decomposition of EOX leads to an activated, short half-life halogenating agent X+ or derivative thereof (e.g., HOX, X₂ or X₃⁻). However, the lack of substrate specificity and lack of regiospecificity exhibited by this class of halogenases strongly argues that halogenation takes place outside the active site and is catalyzed by one of the decomposition products of EOX (Franssen MCR [1994] Halogenation and oxidation reactions with haloperoxidases. Biocatalysis 10:87-11 1).

Non-heme haloperoxidases are of two types, those that possess vanadium, and those that possess a Ser/Asp/His catalytic triad characteristic of serine proteinases. The former group catalyze the vanadium and hydrogen peroxide-dependent formation of HOX which again results in halogenation outside the active site and a pronounced lack of substrate specificity (Franssen MCR [1994] Halogenation and ox idation reactions with haloperoxidases. Biocatalysis 10:87-111). The non-vanadium containing non-herme haloperoxidases are hypothesized to form an acetate ester at the site active Ser residue, which is then converted to peracetic acid in the presence of hydrogen peroxide; peracetic acid oxidizes the halide ion to an activated halogenating species (Pelletier I, Altenbucher J, Mattes R [1995]. A catalytic triad is required by the non-heme haloperoxidase to perform halogenation. Biochim Biophys Acta 1250:149-157). Again, the result is a reaction which fails to proceed with either substrate specificity or regiospecificity van Pee K -H [1996] Biosynthesis of halogenated metabolites by bacteria. Annu Rev Microbiol 50:375-99).

Recently an additional class of halogenase genes has been described whose products exhibit the ability to carry out the regiospecific halogenation of a wide array of natural products (Hammer P E, Hill D S, Lam S T, Van Pee K H, Ligon J M [1997] Four genes from Pseudomonas fluorescens that encode the biosynthesis of pyrrolnitrin. Appl Environ Microbiol 63:2147-2154.

The present invention describes methods of transferring a halogen to a substrate in a regiospecific manner comprising contacting the substrate with a regiospecific halogenase in the presence of an oxidant, a halogen donor, an electron transferase, and a reductant where if the transfer occurs in vivo the electron transferase is encoded by a heterologous nucleic acid molecule.

In particular, methods are described

wherein the method according to the invention further comprises a FAD or FMN component, particularly FAD

wherein the electron transferase is an enzyme capable of catalyzing the electron transfer from NADH or NADPH or ferredoxin to FAD

wherein the electron transferase is an enzyme capable of catalyzing the electron transfer from NADH or NADPH or ferredoxin to the regiospecific halogenase

wherein the electron transferase is a flavin reductase, ferrodoxin NADP reductase, ferredoxin, diaphorase-sufhydryl reductase or NADH-cyt-B5 reductase, NADPH-FMN reductase, NADPH-cyt-p450 reductase or nitrate reductase

wherein the electron transferase comprises an amino acid sequence having at least 30% identity to any one of the amino acid sequences according to SEQ ID NOs: 19, 21, 23, 25, 27, 29 or 31

wherein the electon transferase comprises an amino acid sequence of any one of SEQ ID NOs: 19, 21, 23, 25, 29 or 31

wherein the regiospecific halogenase is prnA, prnC, pyoluteorin halogenases pltA, pltD, and pltM, tetracycline halogenase cts4, hydrolase a, or balhimycin halogenase bha A

wherein the regiospecific halogenase comprises SEQ ID NO: 1

wherein the regiospecific halogenase is a polypeptide comprising an amino acid domain according to any one of SEQ ID NOs: 3, 5, 7, 9, 11, 13, 15 or 17.

Further are provided host cells expressing a heterologous nucleic acid substantially similar to any one of SEQ ID NOs. 18, 10, 22, 24, 26, 28, or 30 and at least one heterologous nucleic acid substantially similar to anyone of SEQ ID NOs: 2, 4, 6, 8,10, 12, 14 or 16, in particular wherein

the host cell is a bacterial, fungal or plant cell

host cell is a microbial cell

the host cell further expresses nucleic acid sequences encoding prnB and prnD

Further are provided

methods of producing pyrrolnitrin comprising growing the host cell as mentioned hereinbefore

methods of protecting a plant against a pathogen comprising treating the plant with said host cell, whereby pyrrolnitrin is produced by the host in amounts that inhibit the pathogen methods as mentioned hereinbefore, further comprising collecting pyrrolnitrin from the host.

Further are provided

plants comprising a host cell according to the invention

methods of protecting a plant against a pathogen, comprising growing the plant as mentioned hereinbefore, whereby pyrrolnitrin is produced in the plant in amounts that inhibit the pathogen

seeds of the plant as mentioned hereinbefore

methods of preventing fungal growth on a crop, comprising growing the plant according to the invention, wherein the plant is a crop plant

methods for improving production of halogenated substrates by a host comprising expressing a heterologous nucleic acid molecule encoding electron transferase in a host wherein the host expresses at least one endogenous polypeptide having regiospecific halogenase activity.

In the present invention it was surprisingly found that regiospecific halogenases are able to transfer a halogen to a substrate in vitro but in order to do so they require an additional protein factor, an electron transferase. The discovery that an additional proteinaceous factor is required to effect halogenation in vitro by these enzymes was made through the purification of PrnA, a D-tryptophan halogenase that functions in the biosynthesis of pyrrolnitrin, a dichlorinated nitrophenylpyrrole antibiotic, by Pseudomonas fluorescens. Purification of this NADH- and flavin adenine dinucleotide (hereinafter “FAD”)-dependent halogenase was accompanied by a progressive decrease in halogenating activity. During ion exchange chromatography of extracts from P. fluorescens overexpressing PrnA, partially purified and inactive PrnA could be reactivated by addition of aliquots of chromatographic fractions from which PrnA was absent. The factor responsible for reactivation herein designated P. fluorescens P2, was subsequently shown to be a protein on the basis of its heat and protease sensitivity. Purification of PrnA to homogeneity led to a complete loss of activity, which could be restored by addition of an electron transferase of the invention.

A second halogenase in the pyrrolnitrin pathway, PrnC, exhibits sequence similarity with PrnA, albeit less sequence similarity to PrnA than to the following regiospecific halogenases known to be involved in biosynthesis of halogenated natural products: pyoluteorin (see, Nowak-Thompson B, Chaney N, Wing J S, Gould S J, Loper J E, [1999] Characterization of the pyoluteorin biosynthetic gene cluster of Pseudomonas fluorescens Pf-5. J Bacteriol 181:2166-2174); chloroeremomycin (see, van Wageningen A M, Kirkpatrick P N, Williams D H, Harris B R, Kershaw J K, Lennard N J, Jones M, Jones S J, Solenberg P J [1998] Sequencing and analysis of genes involved in the biosynthesis of a vancomycin group antibiotic. Chem Biol 5:155-162); balhimycin (see, Peizer S, Sussmuth R, Heckmann D, Recktenwald J, Huber P, Jung G, Wohlleben W [1999] Identification and analysis of the balhimycin biosynthetic gene cluster and its use for manipulating glycopeptide biosynthesis in Amycolatopsis mediterranei DSM5908. Antimicrob Agents Chemother 43:1565-73 and Peizer S, Reichert W, Huppert M, Heckmann D, Wohlleben W [1997] Cloning and analysis of a peptide synthetase gene of the balhimycin producer Amycolatopsis mediterranei DSM5908 and development of a gene disruption/replacement system. J Biotechnol 56:115-128); and chlorotetracycline (see, Dairi T, Nakano T, Mizukami T, Aisaka K, Hasegawa M, Katsumata R [1995] Conserved organization of genes for biosynthesis of chlortetracycline in Streptomyces strains. Biosci Biotechnol Biochem 59:1360-1361, and Dairi T, Nakano T, Aisaka K, Katsumata R, Hasegawa M [1995]Cloning and nucleotide sequence of the gene responsible for chlorination of tetracycline. Biosci Biotechnol Biochem 59:1099-106). Similar to PrnA, purification of PrnC was accompanied by a loss of halogenating activity, which could be restored by the addition of an electron transferase of the invention.

The pyrrolnitrin pathway had previously been shown to function in E. coli when the pyrrolnitrin operon encoding PrnA, PrnB, PrnC and PrnD (for nucleic acid sequence of the pyrrolnitrin operon please see 5.8 X/N, cited in U.S. Pat. No. 5,723,759 which is herein incorporated by reference in its entirety) was expressed. PrnA and PrnC function as halogenases; PrnB catalyzes rearrangement of the indolyl moiety of tryptophan to the aminophenylpyrrole, and PrnD oxidizes the aminophenyl moiety to a nitrophenyl substituent. Surprisingly in the present invention it was found that when an electron transferase of the invention, E. coli flavin reductase (hereinafter “Fre”) in this case, is overexpressed, in vivo production of pyrrolnitrin is significantly enhanced.

The presence of “P2 like activity” was established in E. coli by addition of E. coli extract to purified inactive PrnA. The E. coli P2 like activity was then partially purified by ion exchange, hydroxyapatite, and gel permeation column chromatography. Column fractions containing the activity, and flanking inactive fractions were trypsinized and sequenced by mass spectrometry; the peptides identified in the inactive fractions were subtracted from those present in the active, E. coli P2 like activity containing fraction, and the remaining peptides referred to the E. coli genome database. From this, one nucleic acid sequence was uniquely identified, an NADH-dependent flavin reductase, (hereinafter “fre”, Genbank accession 23486).

As will be described more specifically in the detailed description below, E. coli fre was then cloned and overexpressed, and overexpressing cells shown to possess increases in E. coli P2 like activity directly proportional to their increase in flavin reductase activity. fre was also co-transformed into E. coli along with the pyrrolnitrin operon on separate plasmids. Cells harboring both plasmids produced a significantly higher pyrrolnitrin or pyrrolnitrin metabolites than those harboring the pyrrolnitrin operon alone, confirming the identity of Fre as the accessory factor for PrnA and PrnC, as well as indicating that, in E. coli, flavin reductase activity is a major factor limiting pyrrolnitrin production.

In one embodiment of the invention a method of transferring a halogen to a substrate in a regiospecific manner comprising contacting the substrate with a regiospecific halogenase in the presence of an oxidant, a halogen donor, an electron transferase, and a reductant where if the transfer occurs in vivo the electron transferase is heterologous to the host is provided.

In another embodiment of the invention a method of transferring a halogen to a substrate in a regiospecific manner comprising contacting the substrate with a regiospecific halogenase in the presence of an oxidant, a halogen donor, an electron transferase, a reductant and FAD or FMN, where if the transfer occurs in vivo, the electron transferase is heterologous to the host is provided. In a particularly preferred embodiment, the reaction results in the production of pyrrolnitrin.

In one preferred embodiment the electron transferase is an enzyme capable of catalyzing the electron transfer from NADH or NADPH or ferredoxin to FAD or electron transferase is an enzyme capable of catalyzing the electron transfer from NADH or NADPH or ferredoxin to the regiospecific halogenase.

In one preferred embodiment of the invention, the electron transferase amino acid sequence is at least 30% identical, preferably 40% identical, more preferably 50% identical, more preferably 60% identical, more preferably 70% identical, more preferably 80% identical, or more preferably 90% identical to NADPH-FMN reductase, rat liver NADPH cytochrome P-450 reductase, spinach ferredoxin NADP reductase, cytochrome b5 reductase, or nitrite reductase.

In one preferred embodiment of the invention, the regiospecific halogenase amino acid sequence is at least 30% identical, preferably 40% identical, more preferably 50% identical, more preferably 60% identical, More preferably 70% identical, more preferably 80% identical, or more preferably 90% identical PrnA, PrnC, pyoluteorin halogenases PltA, pltD, and pltM from Pseudomonas fluorescens, tetracycline halogenase cts4 from Streptomyces aurofaciens, hydrolase a from Amycolatopsis orientalis, or balhimycin halogenase bha A from Amycolatopsis mediterranei.

In one preferred embodiment a host cell expressing a heterologous nucleic acid substantially similar to that of an electron transferase of the invention and expressing a heterologous nucleic acid encoding a regiospecific halogenase of the invention is provided. In one preferred embodiment the host cell is a bacterial, fungal or plant cell.

In one preferred embodiment, a host cell expressing heterologous nucleic acid molecules encoding prnA, prnB, prnC, prnD and fre is provided.

In one preferred embodiment a method of producing pyrrolnitrin is provided by growing a host cell, which may include a plant cell, expressing heterologous nucleic acid molecules encoding prnA, prnB, prnC, prnD and fre is provided.

In one preferred embodiment, a plant comprising a host cell expressing a heterologous nucleic acid substantially similar to that of an electron transferase of the invention and expressing a heterologous nucleic acid encoding a regiospecific halogenase of the invention is provided.

In one preferred embodiment, a plant expressing heterologous nucleic acid molecules encoding prnA, prnB, prnC, prnD and an electron transferase of the invention is provided.

### BRIEF DESCRIPTION OF THE SEQUENCE LISTING

SEQ ID NO: 1 is the conserved amino acid motif present in regiospecific halogenases of the invention.

SEQ ID NO: 2 is the nucleic acid sequence encoding PrnA from P. fluorescens.

SEQ ID NO: 3 is the amino acid sequence of PrnA from P. fluorescens

SEQ ID NO: 4 is the nucleic acid sequence encoding PrnC from P. fluorescens

SEQ ID NO: 5 is the amino acid sequence of PrnC from P. fluorescens

SEQ ID NO: 6 is the nucleic acid sequence encoding PltA from P. fluorescens

SEQ ID NO: 7 is the amino acid sequence of PltA from P. fluorescens

SEQ ID NO: 8 is the nucleic acid sequence encoding PltD from P. fluorescens

SEQ ID NO: 9 is the amino acid sequence of PltdD from P. fluorescens

SEQ ID NO: 10 is the nucleic acid sequence encoding PltM from P. fluorescens

SEQ ID NO: 11 is the amino acid sequence of PltM from P. fluorescens

SEQ ID NO: 12 is the nucleic acid sequence encoding hydrolase A from A. orientalis

SEQ ID NO: 13 is the amino acid sequence of hydrolase A from A. orientalis

SEQ ID NO: 14 is the nucleic acid sequence encoding cts4 from S. aureofaciens

SEQ ID NO: 15 is the amino acid sequence of cts4 of S. aureofaciens

SEQ ID NO: 16 is the nucleic acid sequence encoding bhaA from A. mediterranei

SEQ ID NO: 17 is the amino acid sequence of bhaA from A. mediterranei

SEQ ID NO: 18 is the nucleic acid sequence encoding Fre from E. coli

SEQ ID NO: 19 is the amino acid sequence of Fre from E. coli

SEQ ID NO: 20 is the nucleic acid sequence encoding NADH cytochrome b5 reductase from rat.

SEQ ID NO: 21 is the amino acid sequence of NADH cytochrome b5 reductase from rat.

SEQ ID NO: 22 is the nucleic acid sequence encoding NADPH-cyt-p450-reductase from rabbit.

SEQ ID NO: 23 is the amino acid sequence of NADPH-cyt-p450-reductase from rabbit.

SEQ ID NO: 24 is the nucleic acid sequence encoding ferodoxin from S. oleracea.

SEQ ID NO: 25 is the amino acid sequence of ferodoxin from S. oleracea

SEQ ID NO: 26 is the nucleic acid sequence encoding NADPH-FMN reductase from V. Fischeri.

SEQ ID NO: 27 the amino acid sequence of NADPH-FMN reductase from V. Fischeri.

SEQ ID NO: 28 is the nucleic acid sequence encoding ferredoxin-NADP reductase from S. oleracea

SEQ ID NO: 29 is the amino acid sequence of ferredoxin-NADP reductase from S. oleracea

SEQ ID NO: 30 is the nucleic acid sequence encoding nitrate reductase from A. parasiticus

SEQ ID NO: 31 is the amino acid sequence encoding nitrate reductase of A. parasiticus

SEQ ID NO: 32 is the primer for E. coli flavin reductase

SEQ ID NO: 33 is the primer for E. coli flavin reductase

SEQ ID NO: 34 is the plasmid pNOV523

SEQ ID NO: 35 is the plasmid pNOV524

Production of Halogenated Natural Products in Vitro.

According to the present invention, halogenated natural products may be produced in vitro by reacting a regiospecific halogenase with a substrate in the presence of a halogen donor, an oxidant, a reductant, and an electron transferase of the invention.

A regiospecific halogenase of the invention is a halogenase that is capable of interacting with a halide, an oxidant, and a reducing system to catalyze the replacement of one or more carbon-hydrogen bond by one or more carbon-halogen bonds during a biological halogenation reaction and is substrate and/or regiospecific. Regiospecific means that carbon-halogen bonds are formed only at specific locations in a substrate.

Preferred regiospecific halogenases of the invention comprise those that include the following conserved motif and catalyze the replacement of at least one carbon-hydrogen bond by a carbon-halogen bond at a specific location.

x1-W-x2-W-x3-I—P-x4 (SEQ ID NO: 1) where

X1 is G or T;

X2 is V,L,T,F or M;

X3 is any amino acid residue

X4 is I,F,M or L

In a preferred embodiment, the halogenases of the present invention comprise Tryptophan halogenases. Tryptophan halogenases of the invention include PrnA (SEQ ID NO: 3 (see, protein accession #AAB97504; Hammer P E, Burd W, Hill D S, Ligon J M, van Pee K, “Conservation of the pyrrolnitrin biosynthesis gene cluster among six pyrrolnitrin-producing strains. ” FEMS Microbiol Lett Nov. 1, 1999;180(1):39-44) and regiospecific halogenases preferably having 90% identity, 80% identity, 70% identity, 60% identity, 50% identity or 40 % identity to SEQ ID NO: 3. Percent identity between amino acid sequences as used throughout this application is determined by the BASTP 2.09 program available at http://www.ncbi.nlm.nih.gov/gorf/bl2.html where the parameter settings are: blosum62 scoring matrix with a gap opening penalty of 7 and a gap extension penalty of 2 and x_dropoff of 50 and expect of 10. 00 and wordsize of 3.

In another preferred embodiment the regiospecific halogenases of the invention comprise monochchloroaminopyrrolnitrin halogenases. Monochchloroaminopyrrolnitrin halogenases comprise PrnC (SEQ ID NO: 5) having protein accession number AAB97506 and regiospecific halogenase preferably having 90% identity thereto, 80% identity thereto, 70% identity thereto, 60% identity,50% or 40% identity thereto.

In a particularly preferred embodiment of the invention, the regiospecific halogenases of the invention comprise any that are 30% identical, prefereably 40% identical, more preferably 50% identical, more preferably 60% identical, more preferably 70% identical, more preferably 80% identical, more preferably 90% identical, more preferably 95% identical, or more preferably 99% identical to any of prnA (SEQ ID NO: 3), prnC (SEQ ID NO: 5), pyoluteorin halogenases plta (SEQ ID NO: 7), pltD (SEQ ID NO: 9), and pltM (SEQ ID NO: 11) from Pseudomonas fluorescens, tetracycline halogenase cts4 (SEQ ID NO: 15) from Streptomyces aurofaciens, hydrolase a (SEQ ID NO: 13) from Amycolatopsis orientalis, balhimycin halogenase bha A (SEQ ID NO: 17) from Amycolatopsis mediterranei including those identified in the following table:

| | Protein | | |
|---|---|---|---|
| Accession# | Accession | Name | Organism |
| PFU74493_1 | AAB97504 | PrnA | Psuedomonas fluorescens |
| | | | 134 |
| AF161184_1 | AAD46365 | PrnA | Pseudomonas fluorescens |
| | | | CHAO |
| AF161182_1 | AAD46360 | PrnA | Pseudomonas aureofaciens |
| AF161186_1 | AAD46370 | PrnA | Burkholderia pyrrocinia |
| AF161183_1 | AAD46361 | PrnA | Burkholderia cepacia |
| AF161185_4 | AAD46369 | PrnA | Myxococcus fulvus |
| PFU74493_3 | AAB97506 | PrnC | Psueodomonas fluorescens |
| | | | 134 |
| AF161183_3 | AAD46363 | PrnC | Burkholderia cepacia |
| AF161186_3 | AAD46372 | PrnC | Burkholderia pyrrocinia |
| AF161185_2 | AAD46367 | PrnC | Myxococcus fulvus |
| STMCTS_3 | BAA07389 | cts4 | Streptomyces aureofaciens |
| | | tetracycline halogenase | |
| AF081920 | AAD24884 | PltA | Pseudomonas fluorescens |
| AF081920 | AAD24878 | PltD | Pseudomonas fluorescens |
| AF081920 | AAD24882 | PltM | Pseudomonas fluorescens |
| AOPCZA361_2 | CAA11780 | non-heme | Amycolatopsis orientalis |
| | | oxygenase/halogenase | |
| AMOXYAE_4 | CAA76550 | bhaA | Amycolatopsis mediterranei |
| U84350 | AAB49297 | hypothetical | Amycolatopsis orientalis |
| | | hydroxylase a | |

An electron transferase of the invention may comprise an electron transferase capable of transferring electrons from NADH or NADPH or ferredoxin or other reductant to FAD or FMN, or an electron transferase capable of transferring electrons from NADH or NADPH or ferredoxin or other reductant to the halogenase by an NAD(P)H-dependent oxidoreductase or an oxidoreductase with other electron donors, such as the chloroplast photosystem, lactate, xanthine, etc.

Electron transferases of the invention may be determined by selecting electron transferases in which electron transfer can be detected by monitoring oxidation of NADH or NADPH or ferredoxin by the characteristic change in absorbance associated with oxidation of the reductant. This change (or increase in the rate of change) is dependent on the presence of FAD or FMN. Oxidation of NADH and NADPH may be detected by monitoring absorbance at 340 nm; oxidation results in a decrease in absorbance. Oxidation of ferredoxin may be detected by monitoring absorbance at 420 nm; oxidation results in an increase in absorbance. Electron transfer can also be detected by monitoring oxidation of NADH or NADPH by the characteristic decrease in fluorescence with excitation at 340 nm and emission at >380 nm. This decrease in fluorescence is dependent on the presence of FAD or FMN.

Electron transferases of the invention also may be determined by selecting electron transferases in which electron transfer to the regiospecific halogenase of the invention from NADH or NADPH can be identified by mixing the electron transferase with 50 micromolar NADH or 50 micromolar NADPH with or without 50 micromolar halogenase (the halogenase needs to be in the holoenzyme state, that is with all necessary cofactors, such as FAD, already bound) and observing an increase in the rate of oxidation of NADH or NADPH that is dependent on the halogenase; oxidation is measured by a decrease in absorbance at 340 nm or a decrease in fluorescence as described above.

Electron transferases of the invention may be determined by selecting electron transferases in which electron transfer to the halogenase from ferredoxin can be identified by mixing the electron transferase with 50 micromolar reduced ferredoxin with or without 50 micromolar halogenase (the halogenase needs to be in the holoenzyme state, that is with all necessary cofactors, such as FAD, already bound) and observing an increase in the rate of oxidation of that is dependent on the halogenase; oxidation of ferredoxin is measured by an increased absorbance at 340 nm.

In a preferred embodiment of the invention, the electron transferase is least 30% identical, preferably 40% identical, more preferably 50% identical, more preferably 60% identical, more preferably 70% identical, more preferably 80% identical, more preferably 90% identical or identical to any of the following: an E. coli flavin reductase comprising the amino acid sequence of SEQ ID NO: 19 (described by Fieschi F, Niviere V, Frier C, Decout J L, Fontecave M. “The mechanism and substrate specificity of the NADPH:flavin oxidoreductase from Escherichia coli.” J Biol Chem Dec. 22, 1999;270(51):30392-400); diaphorase-sulfhydryl reductase purified according to Richarme, G. “Purification of a new dihydrolipoamide dehydrogenase from Escherichia coli,” J Baxteriol (1989 December ) 171(12): 680-5; NADH cytochrome-b5-reductase (SEQ ID NO: 21) (described by Barber M J, Quinn G B “High-level expression in Escherichia coli of the soluble, catalytic domain of rat hepatic cytochrome b5 reductase.” Protein Expr Purif Aug. 8, 1996(1):41-7); NADPH-cyt-P450 reductase (SEQ ID NO: 23) from rabbit, ferredoxin-NADP reductase (SEQ ID NO: 29) from S. oleracea, ferredoxin (SEQ ID NO: 25) from S. oleracea, nitrate reductase (SEQ ID NO: 31) from A. parasiticus, and NAD(P)H—FMN reductase (SEQ ID NO: 27) from V. fisheri (described by Zenno S, Saigo K “Identification of the genes encoding NAD(P)H-flavin oxidoreductases that are similar in sequence to Escherichia coli Fre in four species of luminous bacteria: Photorhabdus luminescens, Vibrio fischeri, Vibrio harveyi, and Vibrio orientalis.” J Bacteriol 1994 June;176(12):3544-51);. Electron transferases of the invention may be used in extract or purified form.

In a particularly preferred embodiment, the electron transferase of the invention is least 30% identical, preferably 40% identical, more preferably 50% identical, more preferably 60% identical, ore preferably 70% identical, more preferably 80% identical, or more preferably 90% identical to any of SEQ ID NOs: 21, 23, 25 ,27, 29, or 31 and tests positive for electron transfer in any one of the above described tests.

The choice of reductant such as a pyridine nucleotide, eg., reduced nicotinamide adenine dinucleotide or reduced nicotinamide adenine dinucleotide phosphate or reduced ferredoxin depends on the choice of electron transferase of the invention. In general all of the electron transferases of the invention have higher catalytic activity with one or the other pyridine nucleotide; but generally have some activity with the other pyridine nucleotide. Thus, if desirable because of other considerations, the non-preferred pyridine nucleotide may be used in halogenation reactions with particular electron transferases. The preferred pyridine nucleotides of each electron transferase are as follows: NADPH is the preferred pyridine nucleotide for NADPH-cyt-P450 reductase and ferredoxin NADP reductase. NADH is the preferred pyridine nucleotide for E. coliflavin reductase, NADH-cytochrome-b5-reductase, nitrate reductase and diaphorase sulfhydryl reductase.

Ferredoxin NADP reductase can also use reduced ferredoxin which may be generated by illumination of plants, of isolated chloroplasts or of photosystem I containing chloroplast fragments. Ferredoxin may also be reduced by ferredoxin dependent dehydrogenases such as pyruvate: ferredoxin oxidoreductase. (Horner D S, Hirt R P, Embley T M “A single eubacterial origin of eukaryotic pyruvate: ferredoxin oxidoreductase genes: implications for the evolution of anaerobic eukaryotes.” Mol Biol Evol 1999 September;16(9):1280-91).

In a preferred embodiment, FAD may be included in the in vitro reaction to increase efficiency of the reaction. In a particularly preferred embodiment the reaction includes FAD and the selected regiospecific halogenase is PrnA.

In an alternate embodiment, the invention comprises combining the halogenase, where the halogenase is a purified regiospecific halogenase of the present invention with the substrate, a halogen ion such as Cl— and with an active oxygen donor such as H2O2, KlO4, iodosobenzene, iodosobenzoate, tert-butyl hydroperoxide, benzoyl peroxide, cumene hydroperoxide, dicumyl peroxide, peroxy acetic acid or allied compounds. The active oxygen donors dispense with the need to supply O₂ and reductant.

A substrate of the invention will depend on the selected regiospecific halogenase of the invention. Substrates of the invention may include tryptophan, indole, aminophenylpyrrole and derivatives there of and tetracycline, substrates for bhaA including all compounds of the balhimycin substrate classes B1-1, B1-2, B2-1, B2-2 and B3 (described by Pelzer S, Sussmuth R, Heckmann D, Recktenwald J, Huber P, Jung G, Wohlleben W[1999] Identification and analysis of the balhimycin biosynthesis gene cluster and its use for manipulating glycopeptide biosynthesis in Amycolatopsis mediterranei DSM5908. Antimicrob Agents Chemother 43:1565-73)

A halogen donor useful in the present invention, may be supplied to the reaction as a salt of an inorganic or organic cation or as their respective acids. The halogen donor of the invention may provide a F⁻, Cl⁻, Br⁻ or I⁻ ion.

Reactions of the invention may be conducted in buffer with pH between 4 and 10, temperature between 0° C. and 65° C. The halogen donor may be added as a salt, eg. chloride salts may include LiCl, NaCl, KCl, CsCl, MgCl₂ CaCl₂ & NH₄Cl. Reaction times can vary from 1 min to 48 h. Optimal conditions are pH 7. 5, temperature 30° C., reaction time of 12 h.

Efficiency of catalysis in in vitro halogenations may be increased by covalently coupling the electron transferase to the halogenase, thus making electron transfer from the reductant to the halogenase a first order process rather than a second order process (with regard to the concentration of halogenase). The same result can be obtained by genetically engineering a fusion protein containing both an electron transferase and a regiospecific halogenase of the invention by fusing their coding regions in frame. The fusions can be made with or without an intervening sequence coding for a short peptide sequence that separates the electron transferase and halogenase protein domains. Fusion proteins can be made in either of two orientations: (1) N-terminus-electron transferase-(optional linker)-halogenase —C-terminus; (2) N-terminus-halogenase-(optional linker)-electron transferase —C-terminus

In another embodiment of the invention, the protein components of the system comprising a regiospecific halogenase and electron transferase can be immobilized, as described further below and allowed to react with substrates to generate products. The halogenase and electron transferase can be used as individual enzymes that are co-immobilized or as a fusion protein in which the coding sequences for the two components are fused to generate a single protein with electron transferase and halogenase activities. An additional enzyme and appropriate secondary reductant may be included in the system to regenerate NADH or NADPH: examples of such enzyme secondary reductant pairs include: alcohol dehydrogenase and ethanol, glucose-6-phosphate dehydrogenase and glucose-6-phosphate, aldehyde dehydrogenase and acetaldehyde, lipoamide dehydrogenase and reduced thiol such as lipoamide, dithiothreitol or mercaptosulfonic acid.

In this embodiment the enzymes (which would include enzymes of the NADH or NADPH regenerating system if such a system is used) may be immobilized by any of several processes. Examples include: (1) placing the enzymes inside a container with a semipermeable membrane (dialysis membrane) that will allow passage of substrates and nucleotides but not enzymes; (2) covalently attaching the enzymes to an insoluble matrix; (3) binding the enzymes to a matrix via antibodies directed against the enzymes or antibodies directed against antigens fused to the enzymes; (4) binding the enzymes to a matrix via biotin and a biotin-binding domain such as avidin. (5) Polymerizing a matrix (such as a methacrylate polymer) around the enzymes.

The immobilized enzymes may then be exposed to a buffer containing reductant, secondary reductant (if NAD(P)H regenerating system is used), substrate and halide salt. Organic solvents may be included to facilitate solubilization of substrates. Typical conditions comprise pH 4 to 10, 0 to 65° C. After sufficient halogenated product has been generated, the halogenated natural products are removed from the reaction mixture.

Production of Halogenated Natural Products in Heterologous Hosts.

Heterologous nucleic acid molecules encoding an electron transferases of the invention may be expressed in bacterial or fungal hosts to enable the production of the halogenation of natural products with greater efficiency than might be possible from native hosts. For example, to enhance natural product production, a heterologous nucleic acid molecule encoding an electron transferase of the invention may be expressed in pyrrolnitrin producers such as Pseudomonas fluorescens, Burkholderia pyrrocinia, Myxococcus fulvus, Burkholderia cepacia, Pseudomonas aureofaciens, pyoluteorin producers such as Pseudomonas fluorescens, vancomycin class antibiotic producing organisms such as various Amycolatopsis species such as A. orietalis & A. mediterranei and the chlorotetracycline producer Streptomyces aureofaciens, or other antibiotic producing Streptomyces species

Further, heterologous nucleic acid molecules encoding regiospecific halogenases and electron transferases can be co-expressed in bacterial or fungal hosts to enable or increase production of halogenated natural products. In some cases synthesis of the halogenated natural products of the invention will only require one biosynthesis step, the halogenation step and, therefore, the only heterologous nucleic acid molecules that will be expressed will be those comprising coding sequences for the halogenase and electron transferase of the invention. In other cases, one or more halogenation step will be part of a biosynthesis pathway resulting in the halogenated natural product. In this case multiple heterologus nucleic acid molecules will be expressed.

The term “heterologous nucleic acid molecule” as used throughout the present specification refers to a nucleic acid molecule not naturally associated with a host cell into which it is introduced, including genetic constructs, non-naturally occurring multiple copies of a naturally occurring nucleic acid molecule; and an otherwise homologous nucleic acid molecule operatively linked to a non-native nucleic acid molecule.

In its broadest sense, the term “substantially similar”, when used throughout the present specification with respect to a nucleic acid molecule, means a nucleic acid molecule corresponding to a reference nucleotide sequence, wherein the corresponding nucleic acid molecule encodes a polypeptide having substantially the same structure and function as the polypeptide encoded by the reference nucleotide sequence, e.g. where only changes in amino acids not affecting the polypeptide function occur. Desirably the substantially similar nucleic acid molecule encodes the polypeptide encoded by the reference nucleotide sequence. The term “substantially similar” is specifically intended to include nucleic acid molecules wherein the sequence has been modified to optimize expression in particular cells. The percentage of identity between the substantially similar nucleic acid molecule and the reference nucleotide sequence desirably is at least 30%, preferably at least 45%, more desirably at least 65%, more desirably at least 75%, preferably at least 85%, more preferably at least 90%, still more preferably at least 95%, yet still more preferably at least 99% identical. Sequence comparisons are carried out using a Smith-Waterman sequence alignment algorithm (see e. g. Waterman, M. S. Introduction to Computational Biology: Maps, sequences and genomes. Chapman & Hall. London: 1995. ISBN 0-412-99391-0, or at http://www-hto.usc.edu/software/segaln/index.html). The local S program, version 1.16, is used with following parameters: match: 1, mismatch penalty: 0.33, open-gap penalty: 2, extended-gap penalty: 2.

A nucleic acid molecule “substantially similar” to a reference nucleotide sequence hybridizes to the reference nucleotide sequence in 7% sodium dodecyl sulfate (SDS), 0.5 M NaPO₄, 1 mM EDTA at 50° C. with washing in 2× SSC, 0.1% SDS at 50° C., more desirably in 7% sodium dodecyl sulfate (SDS), 0.5 M NaPO₄, 1 mM EDTA at 50° C. with washing in 1× SSC, 0.1% SDS at 50° C., more desirably still in 7% sodium dodecyl sulfate (SDS), 0.5 M NaPO₄, 1 mM EDTA at 50° C. with washing in 0.5× SSC, 0.1% SDS at 50° C., preferably in 7% sodium dodecyl sulfate (SDS), 0.5 M NaPO₄, 1 mM EDTA at 50° C. with washing in 0.1× SSC, 0.1% SDS at 50° C., more preferably in 7% sodium dodecyl sulfate (SDS), 0.5 M NaPO₄, 1 mM EDTA at 50° C. with washing in 0.1× SSC, 0.1% SDS at 65° C. The polynucleotide of the invention that hybridize under the above conditions preferably comprises at least 80 base pairs, more preferably at least 50 base pairs and particularly at least 21, and more particularly 18 base pairs.

Techniques for these genetic manipulations are specific for the different available hosts and are known in the art. For example, the expression vector pKK223 can be used to express heterologous genes in E. coli, either in transcriptional or translational fusion, behind the tac promoter. For the expression of operons encoding multiple open reading frames (hereinafter “ORFs”), the simplest procedure is to insert the operon into a vector such as pKK223 in transcriptional fusion, allowing the cognate ribosome binding site of the heterologous genes to be used. Techniques for overexpression in gram-positive species such as Bacillus are also known in the art and can be used in the context of this invention (Quax et al. In.: Industrial Microorganisms: Basic and Applied Molecular Genetics, Eds. Baltz et al., American Society for Microbiology, Washington (1993)). Alternate systems for overexpression rely on yeast vectors and include the use of Pichia, Saccharomyces and Kluyveromyces (Sreekrishna, In: Industrial microorganisms: basic and applied molecular genetics, Baltz, Hegeman, and Skatrud eds., American Society for Microbiology, Washington (1993); Dequin & Barre, Biotechnology 12:173-177 (1994); van den Berg et al., Biotechnology 8:135-139 (1990)).

Some of these halogenated natural products may be effective in the inhibition of growth of microbes, particularly phytopathogenic microbes. The halogenated natural products can be produced from organisms in which the halogenase and/or electron transferase have been overexpressed, and suitable organisms for this include gram-negative and gram-positive bacteria and yeast, as well as plants which will be described in more detail below. For the purposes of halogenated natural product production, the significant criteria in the choice of host organism are its ease of manipulation, rapidity of growth (i.e. fermentation in the case of microorganisms), and its lack of susceptibility to the halogenated natural product being overproduced. These methods of halogenated natural product production have significant advantages over the chemical synthesis technology usually used in the preparation of halogenated natural products. Application of the methods described here would increase the efficiency and yield in production of halogenated natural products by fermentation and would be useful in introducing new halogen atoms at positions that previously were not present in the natural product and that would be difficult to achieve synthetically.

Some of the advantages over chemical synthesis are cheaper cost of production, and the ability to synthesize compounds of a preferred regiospecificity of halogenation. Incorporation of an electron transferase can increase the efficiency and yield of halogenated products. In addition, novel halogenated products can be produced by addition of halogen to known natural products either by use of naturally occurring halogenases with desired substrate and regiospecificity or by use of engineered halogenases with novel substrate and regiospecificity. It would be very difficult to use chemical means to halogenate many natural products for example, macrolides, polyketides and non-ribosomal peptides, with regiospecificity and enantiomeric specificity. The conditions required for halogenation of aryl or alkyl moieties would generally cause other changes in the structure of the natural product.

Halogenases can also produce enantiomerically pure products (in the case of halogenation of a pro-chiral carbon), as opposed to the racemic mixtures commonly generated by organic synthesis. The ability to produce stereochemically appropriate compounds is particularly important for molecules with many chirally active carbon atoms. Halogenated natural products produced by heterologous hosts can be used for numerous purposes including medical (i.e. control of pathogens and/or infectious disease) as well as agricultural applications.

Where a production of a halogenated product requires more than one enzyme, the nucleic acid molecules encoding enzymes for biosynthesis of the halogenated product of interest may be expressed in a single organism. In one preferred embodiment, all required nucleic acid sequences encoding the enzymes for the natural product would be integrated into the chromosome of the organism as a single operon and controlled by a suitable regulatory element. In an alternate preferred embodiment the nucleic acid sequences could be carried on a plasmid with a selectable marker. Another alternate preferred embodiment comprises expressing the required nucleic acid sequences on two or more compatible plasmids or the required nucleic acid sequences could be distributed among the chromosome and one or more compatible plasmids. Expression of the nucleic acid molecules could be controlled by the native regulatory elements of the natural product biosynthesis nucleic acid coding sequences or by promoters chosen to allow more precise control of the expression of the nucleic acid sequences of the pathway. Optimally, the electron transferase nucleic acid sequences would be included in the operon along with those encoding the regiospecific halogenase (or halogenases) of the invention. Alternatively, the electron transferase sequences may be expressed separately.

Another method of the invention for creating halogenated products comprises dividing nucleic acids molecules from the biosynthesis pathway between two or more separate organisms. The organisms may be grown separately with biosynthesis intermediates produced by one culture being transferred to another culture expressing subsequent steps in the pathway of biosynthesis. Alternately the organisms may be co-cultured with intermediates passing from one to another as required. In any of these applications each halogenase requires a suitable electron transferase co-expressed in the same organism and in the same subcellular location.

Novel halogenated products may be produced by introducing a halogenase into an organism that already expresses genes required to produce the nonhalogenated structure of interest. The halogenase may be engineered to have specificity for the specific site in the completed structure or it may have specificity for a component of the structure that is subsequently incorporated into the final structure in the native organism. For example, a halogenase may be engineered to specifically halogenate an amino acid that is subsequently incorporated into a peptide-containing antibiotic. The resulting product may then possess novel halogen modifications at positions not found in the natural product.

In any of the systems described above, significant advantages in efficiency of halogenation may be effected by fusion of the nucleic acid sequences coding for the electron transferase and the regiospecific halogenase such that a fusion protein is generated with both functionalities; such a fusion may result in higher efficiency of electron transfer from the reductant to the halogenase. The electron transferase nucleic acid sequences may be fused to either the 5′ or the 3′ end of the halogenase. A coding sequence for a short linking peptide (linker) may be incorporated into the fusion, separating the coding sequence for the electron transferase and halogenase protein domains; the length of the linker can vary from 1 to 30 amino acid residues in length.

Halogenase and/or electron transferases of the invention can also be expressed in heterologous bacterial and fungal hosts to produce halogenated natural products with the aim of increasing the efficacy of biocontrol strains of such bacterial and fungal hosts. Microorganisms which are suitable for the heterologous overexpression of anti-pathogenic halogenated natural products are all microorganisms which are capable of colonizing plants or the rhizosphere. As such they will be brought into contact with phytopathogenic fungi, bacteria and nematodes causing an inhibition of pathogen growth. These include gram-negative microorganisms such as Pseudomonas, Enterobacter and Serratia, the gram-positive microorganism Bacillus and the fungi Trichoderma and Gliocladium. Particularly preferred heterologous hosts are Pseudomonas fluorescens, Pseudomonas putida, Pseudomonas cepacia, Pseudomonas aureofaciens, Pseudomonas aurantiaca, Enterobacter cloacae, Serratia marscesens, Bacillus subtilis, Bacillus cereus, Trichoderma viride, Trichoderma harzianum and Gliocladium virens

Expression in heterologous biocontrol strains requires the selection of vectors appropriate for replication in the chosen host and a suitable choice of promoter. Techniques are well known in the art for expression in gram-negative and gram-positive bacteria and fungi, and are described elsewhere in this specification.

Production of Halogenated Products in Transgenic Plants

The halogenases and/or electron transferases of the invention are expressed in transgenic plants thus causing the biosynthesis of the selected halogenated natural products in the transgenic plants. In some cases, the halogenated natural products of the invention will only require one biosynthesis step, the halogenation step, and therefore, the only heterologous nucleic acid molecules that will be expressed will be those comprising coding sequences for the regiospecific halogenase and electron transferase of the invention. In other cases, one or more halogenation steps will be part of a biosynthesis pathway resulting in the halogenated natural product. In this case multiple heterologous nucleic acid molecules will be expressed.

As used in this specification a “plant” refers to any plant or part of a plant at any stage of development. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units. Where the halogenated natural product has anti-pathogenic properties, transgenic plants with enhanced resistance to phytopathogenic fungi and bacteria are generated. For their expression in transgenic plants, the nucleic acid molecules encoding the halogenases and/or electron transferases of the invention and adjacent sequences may require modification and optimization.

Although in many cases nucleic acid molecule from other organisms can be expressed in plants at high levels without modification, low expression in transgenic plants may result from nucleic acid molecules having codons which are not preferred in plants. It is known in the art that all organisms have specific preferences for codon usage, and the codons from other organisms can be changed to conform with plant preferences, while maintaining the amino acids encoded. Furthermore, high expression in plants is best achieved from coding sequences which have at least 35% GC content, and preferably more than 45%. Microbial genes which have low GC contents may express poorly in plants due to the existence of ATTTA motifs which may destabilize messages, and AATAAA motifs which may cause inappropriate polyadenylation. In addition, nucleic acid molecules encoding halogenases or electron transferases of the invention can be screened for the existence of illegitimate splice sites which may cause mRNA truncation. All changes required to be made within the coding sequence such as those described above can be made using well known techniques of site directed mutagenesis, PCR, and synthetic gene construction using the methods described in the published patent applications EP 0 385 962, EP 0 359 472, and WO 93/07278. The preferred nucleic acid molecules of the invention may be unmodified, should these be expressed at high levels in target transgenic plant species, or alternatively may be nucleic acid molecules modified by the removal of destabilization and inappropriate polyadenylation motifs and illegitimate splice sites, and further modified by the incorporation of plant preferred codons, and further with a GC content preferred for expression in plants. Although preferred nucleic acid sequences may be adequately expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17: 477-498 (1989)).

For efficient initiation of translation, sequences adjacent to the initiating methionine may require modification. The sequences cognate to the selected nucleic acid molecules may initiate translation efficiently in plants, or alternatively may do so inefficiently. In the case that they do so inefficiently, they can be modified by the inclusion of sequences known to be effective in plants. Joshi has suggested an appropriate consensus translation initiator for plants (NAR 15: 6643-6653 (1987); SEQ ID NO: 15) and Clontech suggests a further consensus translation initiator (1993/1994 catalog, page 210; SEQ ID NO: 16). These consensuses are suitable for use with the nucleic acid molecules of the invention. The sequences are incorporated into the nucleic acid molecule construction, up to and including the ATG (whilst leaving the second amino acid of the selected nucleic acid molecule unmodified), or alternatively up to and including the GTC subsequent to the ATG (with the possibility of modifying the second amino acid of the transgene).

Expression of the nucleic acid molecules encoding the halogenases or electron transferases of the invention in transgenic plants is behind a promoter shown to be functional in plants. The choice of promoter will vary depending on the temporal and spatial requirements for expression, and also depending on the target species. Where the halogenated natural products are anti-pathogenic and protection of plants against foliar pathogens is desired, expression in leaves is preferred; for the protection of plants against ear pathogens, expression in inflorescences (e.g. spikes, panicles, cobs etc.) is preferred; for protection of plants against root pathogens, expression in roots is preferred; for protection of seedlings against soil-borne pathogens, expression in roots and/or seedlings is preferred. In many cases, however, expression against more than one type of phytopathogen will be sought, and thus expression in multiple tissues will be desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleic acid molecules of the invention. Preferred promoters which are expressed constitutively include the CaMV 35S and 19S promoters, and promoters from genes encoding actin or ubiquitin.

The nucleic acid molecules of the invention can also be expressed under the regulation of promoters which are chemically regulated. This enables the halogenated natural product to be synthesized only when the crop plants are treated with the inducing chemicals, and the halogenated natural product biosynthesis subsequently declines. Preferred technology for chemical induction of gene expression is detailed in the published application EP 0 332 104 and U.S. Pat. No. 5,614,395 (incorporated herein by reference). A preferred promoter for chemical induction is the tobacco PR-1a promoter.

A preferred category of promoters is that which is wound inducible. Numerous promoters have been described which are expressed at wound sites and also at the sites of phytopathogen infection. Ideally, such a promoter should only be active locally at the sites of infection, and in this way the anti-pathogenic halogenated natural product only accumulates in cells which need to synthesize it to arrest growth of the invading pathogen. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Preferred tissue-specific expression patterns include green tissue specific, root specific, stem specific, and flower specific. Promoters suitable for expression in green tissue include many which regulate genes involved in photosynthesis and many of these have been cloned from both monocotyledons and dicotyledons . A preferred promoter is the maize PEPC promoter from the phosphoenol pyruvate carboxylase gene (Hudspeth & Grula, Plant Molec. Biol. 12: 579-589 (1989)). A preferred promoter for root specific expression is that described by de Framond (FEBS 290: 103-106 (1991); EP 0 452 269 [1479]) and a further preferred root-specific promoter is that from the T-1 gene provided by this invention. A preferred stem specific promoter is that described in patent application WO 93/07278 and which drives expression of the maize trpA gene.

Preferred embodiments of the invention are transgenic plants producing the halogenated natural product, pyrrolnitrin in a root-specific fashion. In an especially preferred embodiment of the invention the biosynthesis genes for pyrrolnitrin are expressed behind a root specific promoter to protect transgenic plants against the phytopathogen Rhizoctonia. Further preferred embodiments are transgenic plants producing anti-pathogenic halogenated natural products in a wound-inducible or pathogen infection-inducible manner.

In addition to the selection of a suitable promoter, constructions for halogenated natural product production in plants require an appropriate transcription terminator to be attached downstream of the heterologous halogenase and/or electron transferase nucleic acid molecules. Several such terminators are available and known in the art (e.g. tm1 from CaMV, E9 from rbcS). Any available terminator known to function in plants can be used in the context of this invention.

Numerous other sequences can be incorporated into expression cassettes for halogenase and/or electron transferase nucleic acid molecules. These include sequences which have been shown to enhance expression such as intron sequences (e.g. from Adh1 and bronze1) and viral leader sequences (e.g. from TMV, MCMV and AMV).

The production of halogenated natural products in plants requires that the halogenated natural product biosynthesis nucleic acid molecule encoding the first step in the pathway will have access to the pathway substrate. For each individual halogenated natural product and pathway involved, this substrate will likely differ, and so to may its cellular localization in the plant. In many cases the substrate may be localized in the cytosol whereas in other cases it may be localized in some subcellular organelle. As much biosynthesis activity in the plant occurs in the chloroplast, often the substrate may be localized to the chloroplast and consequently the halogenases and electron transferases of the invention are best targeted to the appropriate organelle (e.g. the chloroplast). Subcellular localization of transgene encoded enzymes can be undertaken using techniques well known in the art. Typically, the DNA encoding the target peptide from a known organelle-targeted gene product is manipulated and fused upstream of the required halogenase and electron transferase nucleic acid molecules. Many such target sequence are known for the chloroplast and their functioning in heterologous constructions has been shown. In a preferred embodiment of this invention the nucleic acid molecules required for pyrrolnitrin biosynthesis are targeted to the chloroplast because the pathway substrate tryptophan is synthesized in the chloroplast.

In some situations, the overexpression of nucleic acids required for halogenated natural product production may deplete the cellular availability of the substrate for a particular pathway and this may have detrimental effects on the cell. In situations such as this it is desirable to increase the amount of substrate available by the overexpression of nucleic acid molecules which encode the enzymes for the biosynthesis of the substrate. In the case of tryptophan (the substrate for pyrrolnitrin biosynthesis) this can be achieved by overexpressing the trpA and trpB encoding nucleic acid molecules. A further way of making more substrate available is by the turning off of known pathways which utilize specific substrates (provided this can be done without detrimental side effects). In this manner, the substrate synthesized is channeled towards the biosynthesis of the halogenated natural product and not towards other compounds.

Vectors suitable for plant transformation are described elsewhere in this specification. For Agrobacterium-mediated transformation, binary vectors or vectors carrying at least one T-DNA border sequence are suitable, whereas for direct transfer any vector is suitable and linear DNA containing only the construction of interest may be preferred. In the case of direct transfer, transformation with a single DNA species or co-transformation can be used (Schocher et al. Biotechnology 4: 1093-1096 (1986)). For both direct transfer and Agrobacterium-mediated transfer, transformation is usually (but not necessarily) undertaken with a selectable marker which may provide resistance to an antibiotic (kanamycin, hygromycin or methotrexate) or a herbicide (basta). The choice of selectable marker is not, however, critical to the invention.

Synthesis of a halogenated natural product in a transgenic plant will frequently require the simultaneous overexpression of multiple nucleic acid molecules encoding the halogenated natural product biosynthesis enzymes. This can be achieved by transforming the individual halogenated natural product biosynthesis nucleic acid molecules into different plant lines individually, and then crossing the resultant lines. Selection and maintenance of lines carrying multiple nucleic acid sequences is facilitated if each the various transformation constructions utilize different selectable markers. A line in which all the required halogenated natural product biosynthesis nucleic acid molecules have been pyramided will synthesize the halogenated natural product, whereas other lines will not. This approach may be suitable for hybrid crops such as maize in which the final hybrid is necessarily a cross between two parents. The maintenance of different inbred lines with different heterologous nucleic acid molecules may also be advantageous in situations where a particular halogenated natural product pathway may lead to multiple halogenated natural products, each of which has a utility. By utilizing different lines carrying different alternative nucleic acid sequences for later steps in the pathway to make a hybrid cross with lines carrying all the remaining required nucleic acid molecules it is possible to generate different hybrids carrying different selected halogenated natural products which may have different utilities.

Alternate methods of producing plant lines carrying multiple nucleic acid sequences include the retransformation of existing lines already transformed with a halogenated natural product biosynthesis nucleic acid molecule or molecules (and selection with a different marker), and also the use of single transformation vectors which carry multiple biosynthesis nucleic acid molecules, each under appropriate regulatory control (i.e. promoter, terminator etc. ). Given the ease of DNA construction, the manipulation of cloning vectors to carry multiple biosynthesis nucleic acid molecules is a preferred method.

Another preferred method is to construct a fusion protein as described above of the halogenase of the invention with the electron transferase of the invention and express a nucleic acid encoding such a fusion protein in a transgenic plant of the invention. The nucleic acid molecule encoding the electron transferase may be fused to either the 5′ or the 3′ end of the halogenase encoding nucleic acid molecule. A linker may, optionally, be incorporated into the fusion, separating the electron transferase and halogenase protein domains. In a preferred embodiment the fusion protein comprises a linker composed of (Gly)₆. However, one skilled in the art will recognize that a linker of other suitable lengths and/or composition may also be selected.

In another preferred embodiment production of halogenated natural products in plants may be achieved by direct plastid transformation. Plastid expression, in which genes are inserted by homologous recombination into the several thousand copies of the circular plastid genome present in each plant cell, takes advantage of the enormous copy number advantage over nuclear-expressed genes to permit expression levels that can readily exceed 10% of the total soluble plant protein. In a preferred embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence. Plastid transformation technology is for example extensively described in U.S. Pat. Nos. 5,451,513, 5,545,817, 5,545,818, and 5,877,462 in PCT application no. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Other selectable markers useful for plastid transformation are known in the art and encompassed within the scope of the invention.

In a particularly preferred embodiment of the invention inducible plastid production of pyrrolnitrin is achieved by direct chloroplast transformation of fre, prnA, prnB, prnC, and prnD as an operon under control of the bacteriophage T7 promoter. Inducible expression is achieved by crossing with plants possessing a nuclear construct encoding the T7 RNA polymerase engineered to possess a chloroplast transit peptide and under the control of the PR1 promoter, allowing BTH-inducible expression.

Production of halogenated natural products by the method of the invention may occur in a wide variety of plant cells, including those of gymnosperms, monocots, and dicots. Although the gene can be inserted into any plant cell falling within these broad classes, it is particularly useful in but not limited to crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

Where an allele(s) for a regiospecific halogenase and/or electron transferase of the invention is obtained by direct selection in a crop plant or plant cell culture from which a crop plant can be regenerated, it is moved into commercial varieties using traditional breeding techniques without the needs for genetically engineering the allele and transforming it into the plant.

### EXAMPLES

The following examples serve as further description of the invention and methods for practicing the invention. They are not intended as being limiting, rather as providing guidelines on how the invention may be practiced.

### Example 1

In Vitro Halogenation Reactions with PrnA

A. Activation of PrnA with E. coli Flavin Reductase, P2, Aspergillus Nitrate Reductase, and Cytochrome b5 Reductase,

PrnA is purified by ion exchange chromatography from Pseudomonas fluorescens BL915deltaORF1-4 with plasmid pPEH14 (prnA) described in Kirner et al (Kirner, S. et al J Bacteriol 1998 April;180(7):1939-43). The purified enzyme has negligible activity without addition of the P2 prepared as described in the Background of the Invention above. Protein concentration or the preparation is 0.36 mg/ml.

The assay mixture is prepared containing HEPES buffer pH 7.5 (50 mM), glucose-6-Phosphate (14.3 mM), D-Trp (7 mM ), NaCl (7 mM). Aspergillus niger Catalase is purchased from Sigma Chemical Co. (13 U/ml), bovine erythrocyte superoxide dismutase (hereinafter “SOD”) is purchased from Sigma Chemical Co. (5 U/ml), Leuconostoc mesenteroides glucose-6-Phosphate dehydrogenase purchased from Sigma (5 U/ml), FAD (7 micromolar). Either a NADH dependent mixture or a NADPH mixture is used as indicated below. An NADH-dependent assay mixture is prepared by adding 12 mg of NADH to 4.5 ml of above described assay mixture. A NADPH-dependent assay mixture is prepared by adding 3 mg NADPH to 1 ml of the above described assay mixture.

The reactions 1-7, described below, are set up in polypropylene tubes in triplicate. After mixing PrnA, the indicated assay mixture and electron transferase, samples are vortexed, then mixed by inversion at room temperature. Reactions are stopped 20.5 hours after initiation of the reactions by boiling for 2 min then the samples are prepared for HPLC analysis by ultrafiltation through Microcon 10 membranes (14000×g for 30 min). The HPLC analysis is by Method Set PrnA1 (described below), the injection volume is 50 microliter, and data are collected for the first 6 minutes.

Standards are prepared by mixing 5 or 10 microliter 7-Cl-Trp (1 mM) with sufficient 50 mM HEPES, pH 7.5 to bring the final volume to 200 microliter. D-Trp is eluted at ˜2 min and 7-Cl-trp is eluted at 4.3 min, as indicated by the elution of the authentic D-Trp and 7-Cl-Trp. The quantity of 7-Cl-trp is determined by comparison to a standard curve. Reported activities are the net increase in 7-Cl-Trp after addition of the electron transferase.

HPLC Analytical Method PrnA1 Determination of 7-Cl-Trp

A Waters Alliance HPLC system with photodiode array detector is used. The Waters Alliance HPLC is equipped with a 4.6×50 mm column packed with C18-Silica, particle size 3 micrometer. A gradient elution method designated here as PrnA1 is used. Flow rates are 1 ml/min throughout and absorbance data are collected from 210 to 400 nm with a resolution of 1.2 nm and a sampling rate of 1/s. The system as pre-equilibrated with an 85:15 mixture of water:methanol. After injection of the sample the column is developed with a 6 min gradient from the starting conditions to a 40:60 water:methanol mixture. Then from 6.0 to 7.0 min the concentration of methanol is increased to 100% in a linear gradient. The column is washed for 1 min with 100% methanol, then re-equilibrated. D-Trp eluted at ˜2 min and 7-Cl-trp eluted at 4.3 min, as indicated by the elution of authentic D-Trp and 7-Cl-Trp.

1. Activation of PrnA by E. coli Flavin Reductase

E. coli flavin reductase, (abbreviated herein after as Fre), is purified by ammonium sulfate precipitation followed by hydrophobic chromatography, in a method based on the protocol of Fieschi et al (1995) J. Biol. Chem 270 30392-30400 which is herein incorporated by reference in its entirety. The flavin reductase purification follows the procedure of Fieschi through bacterial homogenization and ammonium sulfate fractionation. At which point flavin reductase activity is precipitated. The precipitate is collected by centrifugation, resuspended in 25 mM Tris/Cl pH 7.5 0.5 M KCl 10% glycerol. The method of Fontcave et al J Biol Chem Sep. 5, 1987;262(25):12325-31 which is herein incorporated be reference in its entirety is then followed to completion. The protein concentration of the of the collected purified Fre sample is 21 microgram/ml. Each reaction contains 20 microliter PrnA, 160 microliters of the NADH mixture described above and 20 microliter of Fre. The resulting net product formation was 21.46±1.02 nmol 7-Cl-Trp.

2. Activation of PrnA by P2

P2 is an electron transferase protein preparation from Pseudomonas fluorescens purified by ion exchange chromatography and described above in the Background of the invention. It has no PrnA activity. Protein concentration of the P2 sample is 4.8 mg/ml. Each reaction contains 20 microliter PrnA, 160 microliter of NADH mixture and 20 microliter of P2. The resulting net product formation was 12.50±2.02 nmol 7-Cl-Trp.

3. Activation of PrnA by Spinach Nitrate Reductase

Recombinant FAD-domain of spinach nitrate reductase (hereinafter “SNIR) (18.6 micromolar). Each reaction contains 20 microliter PrnA, 160 microliter of NADH mixture and 20 microliter of SNIR. The resulting net product formation was 0.048±0.73 nmol 7-Cl-Trp.

4. Activation of PrnA by Aspergillus Nitrate Reductase

Nitrate reductase from Aspergillus sp. (10 U/ml) was purchased from ICN. Each reaction contains 20 microliter PrnA, 160 microliter of NADH mixture and 20 microliter of Nitrate reductase. The resulting net product formation was 1.49±0.18 nmol 7-Cl-Trp.

5. Activation of PrnA by Rat NADH-Cytochrome-b5-Reductase

Recombinant soluble domain of rat hepatic cytochrome b5 reductase (11. 7 micromolar) is obtained. Each reaction contains 20 microliter PrnA, 160 microliter of NADH mixture and 20 microliter of cytochrome b5 reductase. Net product formation was 0.31±0.11 nmol 7-Cl-Trp.

6. Activation of PrnA by Diaphorase Sulfhydryl Reductase

Diaphorase sulfhydryl reductase (200 U/ml) is purchased from United States Biochemicals. Each reaction contains 20 microliter PrnA, 160 microliter of NADH mixture and 20 microliter of diaphorase. Net product formation was 2.24±0.04 nmol 7-Cl-Trp.

7. Activation of PrnA by Rabbit NADPH- cyt-P450 Reductase

Rabbit liver NADPH-cyt-P450 reductase (0.069 mg/ml) is purchased from Sigma Chemical Co. Each reaction contains 20 microliter PrnA, 160 microliter of NADPH mixture and 20 microliter of cytochrome P450 Reductase. The resulting net product formation was 3.35±0.23 nmol 7-Cl-Trp.

### Example 2

Activation of PrnA by E coli Flavin Reductase; Spinach Ferredoxin NADP Reductase, Spinach Ferredoxin NADP Reductase+Spinach Ferredoxin; and Photobacterium fischeri NAD(P)H:FMN Reductase

The following components are used in examples 1-4 below: PrnA (described for Example 1 above) at 0.36 mg/ml, Assay Mixture containing HEPES (100 mM), glucose-6-phosphate, disodium salt (50 mM), D-Trp (5 mM), NaCl (5 mM). Aspergillus niger catalase (39 U/ml), bovine erythrocyte superoxide dismutase (15 U/ml), Leuconostoc mesenteroides glucose-6-phosphate dehydrogenase (10 U/ml). NADH-(3 mg/ml) NADPH (3 mg/ml).

Each assay contains the assay mixture, either NADH (for samples containing Fre and the NAD(P)H:FMN reductase) or NADPH (for samples containing FNR or FNR and Fd), PrnA and the indicated electron transferase. Negative control samples are incubated in parallel; these substituted buffer for PrnA. Quantification standards are prepared by diluting 0,1, 2 or 5 microliter of 7-Cl-Trp standard (1 mM) in 100 microliter Assay Mixture, 50 microliter NADH, 20 microliter PrnA and 50 microliter buffer; the tubes were heated to 100 C. prior to addition of PrnA, then heated an additional 2 min. Further processing is in parallel with the enzymatic reactions. All samples are mixed for 2 h at room temperature. Reactions are stopped and samples processed as described above in Example 1, including using the HPLC Analytical method PrnA1 as described in Example 1.

1. Activity of PrnA with Fre: 100 microliter Assay Mixture, 50 microliter of NADH, 20 microliter PrnA and 50 microliter of Fre (0.84 microgram/ml) are mixed as described above. Net 7-Cl-Trp produced was 8.44 nmol.

2. Activity of PrnA with ferrodoxin NADP reductase: 100 microliter Assay Mixture, 50 microliter of NADH, 20 microliter PrnA and 50 microliter of FNR (4.1 micromolar) are mixed as described above. Net 7-Cl-Trp produced was 4.22 nmol.

3. Activity of PrnA with ferrodoxin NADP reductase and ferrodoxin: 100 microliter Assay Mixture, 50 microliter of NADH, 20 microliter PrnA and 50 microliter of FNR (4.1 micromolar) and Fd (7 micromolar) are mixed as described above. Net 7-Cl-Trp produced was 9.15 nmol.

4. Activity of PrnA with Photobacterium fischeri NAD(P)H:FMN reductase: 100 microliters Assay Mixture, 50 microliter of NADH, 20 microliter PrnA and 50 microliter of NAD(P)H:FMN reductase purchased from Roche (4 U/ml) are mixed as described above. Net 7-Cl-Trp produced was 0.11 nmol.

### Example 3

In Vitro Halogenation Reactions with PrnC

Fre, ferrodoxin NADP reductase, ferrodoxin and NADPH FMN-reductase are tested for the ability to activate P. fluorescens PrnC that was depleted of endogenous electron transferase (P2) as described below. PrnC catalyzes chlorination of monodechloroaminopyrrolnitrin (MDA) to yield aminopyrrolnitrin (APRN).

The following materials are prepared for use in assays described below. Buffer 100 mM Tris/Cl, 1 mM EDTA. pH 7.5. Monodechloroaminopyrrolnitrin (MDA) 74.2 mM is prepared by culture of Pseudomonas fluorescens expressing PrnA and PrnB as described in (Kirner et al,1998) which is herein incorporated by reference in its entirety. The assay mix comprises FAD (5 μM) and MDA (742 μM) in buffer. NADH is dissolved in buffer at a concentration of 6 mg/ml. or NADPH is dissolved in buffer at a concentration of 6 mg/ml. Extract #1 is a crude extract in buffer containing PrnC and the endogenous electron transferase P2 described above in example 1. PrnC is expressed in P. fluorescens bacteria (pPEH/prnC/134Δprn) that had the chromosomal prn operon deleted but comprises a nucleic acid sequence (SEQ ID NO: 4) encoding PrnC behind a tac promoter on plasmid pPEH-PrnC (Kirner et al, 1998). In this system the tac promoter results in constitutive expression of PrnC. Extract #2, PrnC in Extract #1 is purified by mixing Extract #1 with an anion exchange resin then removing the resin by centrifugation. to deplete PrnC of P. fluorescens P2 activity using 100 mM Tris/Cl buffer.

The assays described below are run as follows; Extract #2 is mixed with the indicated electron transferase, assay mix and either NADH or NADPH as indicated. The native activity of PrnC prior to removal of the P2 activity is determined by a parallel sample in which extract #1 is mixed with assay mix and NADH. All samples are mixed by inversion overnight, then reactions are stopped by addition of 10 microliter KOH (6 M) followed by extraction with ethyl-acetate (1 ml). 0.6 ml of the organic soluble layer is removed to a separate tube and the solvent is removed by vacuum centrifugation. The residue is redissolved in 200 microliter of 60/40 H₂O/CH₃CN+100 microliter CH₃CN. The samples are filtered through 0.2 μm nylon filters to remove particulate matter. Samples are analyzed by PrnC_Iso method described below. The 220 nm absorbance chromatogram is analyzed and integrated. PrnC activity is expressed as 100 times the ratio of the APRN peak area to the sum of the peak areas of APRN and MDA. Assuming equal extinction coefficients at 220 nm the calculated ratio is equivalent to the net % conversion of MDA to APRN by halogenation.

HPLC Analytical method PrnC_Iso The HPLC instrument used is a Waters Alliance HPLC system with photodiode array detector and is equipped with a 4.6×50 mm column packed with C18-Silica, particle size 3 micrometer. The HPLC method is an isoctratic elution method in which flow rate is 1.5 ml/min and solvent is a 58:42 ratio of water:acetonitrile. Absorbance data are collected from 210 to 400 nm with a resolution of 2.4 nm and a sampling rate of 5/s. The system is pre-equilibrated for a minimum of 6 min prior to injection. Injection volume is 50 microliter, data collection time 6 min followed by an additional 6 min of isocratic elution before injection of the next sample. MDA is eluted at 2.16 min in this method and aminopyrrolnitrin (APRN) is eluted at 3.05 min.

Protein concentrations are determined by the BCA method using the standard procedures described by the vendor (Pierce).

1. PrnC activity with E. coli Fre: 50 microliter of Extract #2 is mixed with 20 microliter of E. coil flavin reductase, (21 microgram/ml), 100 microliter assay mix, and 50 microliter NADH; mixing continues overnight followed by product analysis as described above. Observed activity of 51.8% conversion of MDA to APRN.

2. PrnC activity with spinach ferrodoxin NADP reductase: 50 microliter of Extract #2 is mixed with 20 microliter of spinach ferredoxin:NADP reductase (20.7 micromolar), 100 microliter assay mix, and 50 microliter NADH; mixing continues overnight followed by product analysis as described above. Observed activity of 1.8% conversion of MDA to APRN.

3. PrnC activity with spinach ferrodoxin NADP reductase and spinach ferrodoxin: 50 microliter of Extract #2 is mixed with 20 microliter of spinach ferredoxin:NADP reductase (20.7 micromolar) and spinach ferredoxin (Fd) (35 micromolar), 100 microliter assay mix, and 50 microliter NADH; mixing continues overnight followed by product analysis as described above. Observed activity of 2.5% conversion of MDA to APRN.

4. PrnC activity with NADPH FMN reductase: 50 microliter of Extract #2 is mixed with 20 microliter of NAD(P)H:FMN reductase (10 U/ml) from Photobacterium fischeri, 100 microliter assay mix, and 50 microliter NADH; mixing continues overnight followed by product analysis as described above. Observed activity of 4.0% conversion of MDA to APRN.

5. Native activity of PrnC prior to removal of the P2 activity is determined by a parallel sample in which Extract #1 (50 microliter) is mixed with assay mix (100 microliter) and NADH (50 microliter); mixing continues overnight followed by product analysis as described above. Observed activity of 7.8% conversion of MDA to APRN.

### Example 4

Halogenation in E. coli

Cloning of Nucleic Acid Encoding E. coli Flavin Reductase.

The nucleic acid sequence encoding E. coli flavin reductase (hereinafter “fre”) is PCR replicated from the E. coli strain XL-1 Blue (Stratagene) using the primers 5′ GCGCGAATTCATGACAACCTTAAGCTGTAMGTGACC (SEQ ID NO: 32) and 3′GCGCCTGCAGTCAGATAAATGCAAACGCATCGCC (SEQ ID NO: 33). The nucleic acid molecule is then Topo cloned (Invitrogen ), transformed into E. coli XL-1 Blue (Stratagene) and transformants are selected by plating onto Luria broth (LB) solid medium supplemented with ampicillin. Several colonies are selected and analyzed by DNA sequencing to confirm their identity. Of these, one was found to possess a nucleic acid molecule comprising a sequence identical to that of the reported fre (Genbank accession 23486). A second possesses a nucleic acid sequence comprising a mutation at nucleotide 247 that resulted in a charged amino acid substitution of Lys83 to Glu83 (mutant hereinafter referred to as fre^{E83}.)

B. Inducible Overexpression of fre and the fre ^{E83}Mutant

Inducible overexpression of fre and the fre^{E83} mutant is accomplished by cloning of wild-type fre and the substitution mutant fre^{E83} into the EcoR1/Pst1 sites of pKK223-3 (Pharmacia) under control of the tac promoter. After transformation cells comprising fre-pKK223-3, fre^{E83} -pKK223-3, and the empty vector pKK223-3 are grown in 6 mL LB +amp at 37 C. overnight, then diluted into 30 mL LB+amp, 5 mM IPTG (Fisher) for 5 hr and harvested by centrifugation. Bacterial pellets are suspended in 4.5 mL 50 mM HEPES pH 7.5, 1 mM EDTA plus 0.5 mL 5 mg/mL lysozyme for 15 min at 25 C., subjected to two freeze-thaw cycles. After sonication for 1 min on ice, homogenates are centrifuged at 16K×g for 20 min. The supernatants are then serially diluted with 50 mM Hepes pH 7.5, 1 mM EDTA to generate 8 samples with relative concentration ranging from 1 to 1/10000.

Each bacterial extract and diluted bacterial extract is assayed for complementation of PrnA activity by addition of 20 microliter of extract to a 180 microliter of a solution composed of 7.2 microgram PrnA (0.36 microgram/microliter), 3.3 micromolar FAD, 3.3 mM NaCl, 1.67 mM D-Trp, 0.67 mg/ml NADH and 50 mM HEPES, pH 7.5. The reactions are incubated at 30 C. for 2 h. The reactions are stopped by heating to 100° C. for 2 min followed by centrifugation at 21000×g for 5 min. The supernatant solutions are then filtered through 10 kDa cutoff centrifugal ultrafiltration membranes. The filtrate is then assayed by reverse-phase HPLC to quantify conversion of D-Trp to D-7-chlorotryptophan using the analytical method, described above in Example 1 for PrnA1. Addition of extract from E. coli containing the empty vector pKK223-3 resulted in 0.34 pmol 7-Cl-Trp per min per microgram protein in the added extract Addition of extract from E. coli containing fre^{E83}-pKK223-3 resulted in 1.14 pmol 7-Cl-Trp per min per microgram protein in the added extract. Addition of extract from E. coli containing fre-pKK223-3 resulted in 301 pmol 7-Cl-Trp per min per microgram protein in the added extract.

Flavin reductase assays are carried out by addition of 10 microliter bacterial extract to 990 microliter 50 mM Hepes pH 7.5 containing 0.1 mg/ml NADPH and 9.5 micromolar riboflavin. If the activity is too high to permit observation of the first 20% of the reaction, the bacterial extract is diluted 1/10 in 50 mM HEPES buffer then assayed as above. Conversion of NADH to NADP is then monitored spectrophotometrically at 340 nm. Addition of extract from E. coli containing the empty vector pKK223-3 had a flavin reductase activity of 0.055 nmol per min per microgram protein in the added extract. Addition of extract from E. coli containing fre^{E83}-pKK223-3 had a flavin reductase activity of 0.157 nmol per min per microgram protein in the added extract. Addition of extract from E. coli containing fre-pKK223-3 had a flavin reductase activity of 25.4 nmol per min per microgram protein in the added extract. This demonstrates that changes in flavin reductase activity is proportional to changes in halogenation activity.

C. Co-Expression of fre and the prn Operon in E. coli.

The complete Pseudomonas fluorescens pyrrolnitrin operon (5.8 X/N, cited in U.S. Pat. No. 5,723,759 which is herein incorporated by reference above) in pKK223-3 (Pharmacia) was transformed in to E. coli. The fre sequence, including the Taq promoter, is transferred from pKK223-3 into the tetracycline marker of pACYCl84 (NEB), which contains the compatible origin of replication p15A. This plasmid is then co-transformed with 5.8X/N and presence of both vectors are selected for by ampicillin and chloramphenicol. The host strain containing fre alone are also generated as a negative control. A 60 mL culture of each line was grown at 37° with shaking at 200 rpm for 48 hr. From each culture 5 mL is extracted for plasmid analysis to confirm the presence of one or both plasmids. A 15 mL aliquot is used for protein and activity analysis. The remaining 40 mL of culture is extracted 2 times with 2 volumes of ethyl acetate. The ethyl acetate fractions are concentrated to dryness in vacuo and then brought up into 50 microliter 6:4 H₂O/CH₃CN and 60 microliter MeOH. Twenty microliter of the resulting solutions are then analyzed by HPLC method Prn_BCD for aminopyrrolnitrin, and pyrrolnitrin described below.

HPLC Analytical Method Prn_BCD Determination of MDA, APRN and PRN.

The HPLC instrument is a Waters Alliance HPLC system with photodiode array detector and is equipped with a 4.6×50 mm column packed with C18-Silica, particle size 3 micromolar. The HPLC method is a gradient elution method. Flow rates are 1.2 ml/min through out and absorbance data are collected from 210 to 400 nm with a resolution of 2.4 nm and a sampling rate of 5/s. The system is pre-equilibrated with 65:35 ratio of water: acetonitrile. Following sample injection the column is developed in a linear gradient from the starting conditions to a 40:60 ratio of water: acetonitrile. Aminopyrrolnitrin is eluted at 5.0 min and pyrrolnitrin at 6.6 min. Both aminopyrrolnitrin and pyrrolnitrin are measured by integrating peak areas in chromatograms measured at diagnostic wavelengths. For aminopyrrolnitrin 300 nm absorbance is used. For pyrrolnitrin, 250 nm absorance is used.

The results show that aminopyrrolnitrin accumulation was increased greater than 10-fold, and pyrrolnitrin accumulation was increased greater than 4-fold, in E. coli cells co-expressing plasmids comprising fre and the pyrrolnitrin operon compared to cells expressing only the pyrrolnitrin operon.

### Example 5

Halogenation by PrnA Expressed in Transgenic Plants then Purified and Assayed in Vitro.

Arabidopsis thaliana, ecotype Columbia, is transformed (by the Agrobacterium-mediated transformation method) with the four nucleic acid molecules of the pyrrolnitrin operon, encoding PrnA, PrnB, PrnC & PrnD, each behind the ubiquitin promoter as described below in Example 6.

The individual pyrrolnitrin nucleic acid molecules are PCR replicated with appropriate restriction sites from pCIB169 (U.S. Pat. No. 5,723,759) which contain a cosmid clone from P. fluorescens BL915, Genbank accession number is U74493. The nucleic acid molecules are subdloned and sequenced. The ubiquitin3 promoter and first intron (J. Callis et al (1990). Journal of Biological Chemistry 265:12486-12493. and S. R. Norris et al (1993) Plant Molecular Biology. 21:895-906. ) are PCR replicated from the Arabidopsis genome to contain a 5′Kpnl and a 3′ BamHl site. The ubiquitin promoter, nos terminator (Depicker et al (1982) Journal of Molecular and Applied Genetics 1:561-573. ) and each individual pyrrolnitrin nucleic acid molecule (see U.S. Pat. Nos. 5,723,759 and 5,955,348 each of which is herein incorporated by reference in its entirety) are cloned into a modified pSport1 vector. A Kozak consensus −3ACC nucleotide triplet is added to each of PrnA, B and D just 5′ of the initial ATG. The PrnC nucleic acid molecule is not modified. The initial GTG codon in PrnB is changed to an ATG codon. These modifications result in the vector set pPEH7826, 27, 28 and 29 (Prn A, B, C, D respectively). All other sequences are consensus to the wild type sequence. The PrnAC doublet is constructed by inserting the Kpnl fragment from pCIB7826 into the Kpnl site of pCIB7828 (PrnC) producing pCIB7830. The PrnBD doublet is produced by inserting the Kpnl fragment from pCIB7827 (PrnB) into the Kpnl site of pCIB7829 (PrnD) producing pCIB7831. The four nucleic acid molecule operon is created by inserting the Notl fragment from pCIB7830 into the Notl fragment of pCIB7831 producing pCIB7832. The Xbal fragment from pCIB7832 was inserted into the binary vector pCIB200 producing the transformation vector pCIB7819. The final vector is electroporated into agrobacterium and used for Arabidopsis transformation.

Arabidopsis thaliana is transformed by the method of N. Bechtold et al (N. Bechtold et al (1993). C. R. Acad. Sci. Paris, Life Sciences 316:1194-1199).

Two transformed lines (3 and 12) and a nontransformed control line are grown and leaves (1 g) harvested. The leaves are frozen in liquid N₂, powdered in a mortar and extracted with 6 ml of LS buffer (50 mM HEPES, pH 7.5, 5 mM NaCl). After centrifugation at 5000×g for 15 min to pellet debris, the supernate is filtered through glass wool to remove residual particles.

PrnA is immunopurified by mixing extract (3 ml) with an affinity matrix. The affinity matrix is prepared by mixing for 30 minutes, at room temperature, 100 microliters of rabbit antigoat-IgG-agarose (purchased from Sigma) with 50 microliters of goat anti-PrnA sera. Then the agarose beads are washed three times with 1 ml of LS buffer. After mixing the 3 ml sample with the affinity matrix, unabsorbed material is removed from the beads by washing with LS buffer. A positive control sample is prepared by mixing 5 microliter of PrnA (0.36 microgram/microliter) purified from Pseudomonas fluorescens as described in Example 1 with 3 ml LS then treating in parallel with the plant extract samples.

200 microliters of assay buffer (50 mM HEPES pH 7.5, 5 mM D-Trp, 5 mM NaCl 5 μM FAD, 5 mM glucose 6-phosphate+2 mg/ml NADH+6.25 U/ml glucose 6-phosphate dehydrogenase+44 U/ml catalase+30 U/ml SOD ) and 20 microliter of Fre (21 microgram/ml) purified from E. coli as described in example 1 are added to the agarose beads containing immunopurified PrnA, except for one sample each of lines 3 and 12. Samples are then mixed overnight by inversion, filtered through Microcon-10 filters, then product assayed by HPLC method PrnA1 (described above in Example 1) The injection volume of sample was 50 microliter. The following levels of 7-Cl-Trp were found: positive control (exogenous PrnA added to non-transformed plant extract) 185 pmol, Line 3+Fre (two separate samples) 83 pmol and 113 pmol, Line 3 without Fre 0 pmol, Line 12 with Fre (two separate samples) 120 pmol and 64 pmol, Line 12 without Fre 0 pmol, nontransformed control 0 pmol.

These data demonstrate that transformed plants express PrnA in an active form whose activity was dependent on the addition of Fre.

### Example 6

Halogenation in Transgenic Plants

A. Cytoplasmic Production of Halogenated Compounds in Transgenic Plants by Transformation of Nucleic Acid Encoding E. coli Flavin Reductase into Plants Comprising Nucleic Acid Encoding PrnA, PrnB,PrnC and PrnD.

The nucleic acid sequence of SEQ ID NO: 6, encoding flavin reductase from E. coli, is cloned into the vector pNOV019, to place the nucleic acid molecule under the control of the Arabidopsis ubiquitin10 (UB10) promoter (J. Callis et al (1990). Journal of Biological Chemistry 265:12486-12493. and S. R. Norris et al (1993) Plant Molecular Biology. 21:895-906.), and terminated with the nopaline synthase terminator from Agrobacterium (Depicker et al (1982) Journal of Molecular and Applied Genetics 1:561-573).

The binary vector system consisting of pNOV507 (Kan^{R}), 508, (Chlor^{R}) and 509, (AmP^{R}) is completed. The three vectors used to construct the pyrrolnitrin operon with the fre nucleic acid molecule and a herbicide resistance selectable marker are as follows. pNov507 (Kan^{R}) is the binary vector with the polylinker between the left and right borders replaced with a selection of unique restriction sites that are not found in any of the promoters, terminator, pyrrolnitrin, fre, or the selectable marker nucleic acid molecules. The other two vectors pNOV508 (ChlorR) and pNOV509 (AmpR) are vectors which contain a portion of the pNOV507 polylinker with additional restriction sites added for cloning the separate nucleic acid molecule cassettes for the pyrrolnitrin operon. These two vectors are construction or assembly vectors. The fre cassette along with the UB3-selectable marker cassette from pNOV111 are ligated together in pNOV509. This double cassette is then transferred into the binary vector, pNOV507, yielding the final vector pNOV510. This vector is electroporated into Agrobacterium. The Arabidopsis thaliana lines that are transformed with PrnA, PrnB, PrnC and PrnD nucleic acid molecules as described in Example 5 are transformed by the method of N. Bechtold et al (N. Bechtold et al (1993). C. R. Acad. Sci. Paris, Life Sciences 316:1194-1199).

All of the pyrrolnitrin pathway nucleic acid molecules in plants and various constructions are driven by the Arabidopsis ubiquitin3 (UB3) promoter (J. Callis et al (1990) Journal of Biological Chemistry 265:12486-12493. and S. R. Norris et al (1993) Plant Molecular Biology. 21:895 -906. ), and terminated with the nos terminator from Agrobacterium. Homozygous Arabidopsis lines harboring prnA, prnB, prnC and prnD, and wild type Columbia, are transformed with pNOV510 by Agrobacterium infiltration by the method of N. Bechtold et al as described above. Seeds are collected, dried down and planted on soil. Transformed plants are identified by spraying the seedlings with the selective agent at 0.025% three times over eight days. Plant are then confirmed for presence and level of pyrrolnitrin by HPLC or gas chromotography-mass spectometry. Plant extracts may also or alternatively be confirmed for prnA and/or prnC activity as described above.

B. Cytoplasmic Production of Halogenated Compounds in Transgenic Plants by Co-Transformation of E. coli Flavin Reductase and the Pyrrolntrin Operon

Nucleic acid sequences encoding PrnA, PrnB, PrnC, and PrnD of the pyrrolnitrin pathway set forth in U.S. Pat. No. 5,723,759 and incorporated by reference above and SEQ ID NO: 7 encoding E. coli flavin reductase are introduced into plants in a single t-DNA construct. Expression of each of the pyrrolnitrin biosynthesis nucleic acid molecules is driven by the UB3 promoter, whereas fre SEQ ID NO: 7 is driven by UB10. All five nucleic acid molecules have been either conformed or altered to conform closely to a Kozak translational initiation sequence by possession of A at −3. All of the nucleic acid molecules are terminated by the nos terminator. In a preferred embodiment, the final vector is constructed by assembling the UB3 promoter-cytosolic targeted pyrrolnitrin biosynthesis genes and the UB10-fre cassettes in a binary vector comprising the following: Right border-UB3-prnA-nos-UB3-prnC-nos-UB3-prnB-nos-UB3-prnD-nos-UB10-fre-nos-UB3-selectable marker-nos-Left Border. This vector is named pNOV523 (SEQ ID NO: 34).

In another embodiment, the cytosolic targeted pyrrolnitrin operon is created by ligating the Notl A/B doublet fragment from pCIB7830 into the C/D doublet vector pCIB7831. The operon is transferred into pNOV507 as an Xbal cassette. The Notl A/B doublet from pCIB10253 is ligated into the C/D doublet vector pCIB10254. This construction is also transferred to pNOV507 as an Xbal cassette.

The final vector contains the following: Right border-UB3-prnA-nos-UB3-prnB-nos-UB3-prnC-nos-UB3-prnD-nos-UB10-fre-nos-UB3-selectable marker-nos-Left Border.

This vector is then electroporated into Agrobacterium and used to transform Arabidopsis (Columbia), by Agrobacterium infiltration (N. Bechtold et al (1993). C. R. Acad. Sci. Paris, Life Sciences 316:1194-1199. ) Seeds are collected, dried down and planted on soil. Transformed plants are identified by spraying the seedlings with the selective agent at 0.025% three times over eight days. Plants are then confirmed for presence and level of pyrrolnitrin by HPLC or gas chromatography-mass spectrometry.

C. Production of Halogenated Compounds in Plastids of Transgenic Plants.

Nucleic acid constructs encoding prnA and B are engineered to express chloroplast transit peptides (Wong, E. Y. et al (1992) Plant Molecular Biology vol. 20: 81-93. ), and placed together on a vector allowing selection on kanamycin. Transformation protocols are as detailed in previous examples (N. Bechtold et al (1993). C. R. Acad. Sci. Paris, Life Sciences 316:1194-1199).

Construction of the plastid targeted pyrrolnitrin nucleci acid molecule vectors.

The individual pyrronitrin pathway nucleic acid molecules are PCR replicated from pCIB10230, 31, 32, 33 (Prn A, B, C, D respectively), to contain a 5′ Nhel and 3′ BamHl restriction sites. The nucleic acid molecules are Topo cloned into pCR2.1 (Invitrogen, US Office Carlsbad, Calif. 92008, catalogue number K2030-01) for sequence confirmation. The RuBPcase small subunit peptide transit sequence, is PCR replicated from the Arabidopsis cDNA library in pFL61 (Wong et al., 1992, Plant Mol Biol 20: 81-93). This nucleic acid sequence is ligated onto the 5′ end of each pyrronitrin nucleic acid molecule in pPEH31, 30, 29, and 28 (Prn A, B, C, D repsectively). This pPEH vector set contains the UB3-intron-nos cassette. The additional mature peptide is synthesized as complimentary oligos, annealed and ligated onto the 5′ portion of the transit peptide pyrrolnitrin nucleic acid molecule construction. This produced the plastid targeted pyrrolnitrin nucleic acid molecule vectors pCIB10249, 50, 51 and 52 (Prn A, B, C, D respectively). The PrnAB doublet pCIB10253, was created by ligating the PrnA containing Kpnl nucleic acid molecule cassette from pCIB10249 into pCIB10250. The PrnCD doublet, pCIB10254 was created by ligating the PrnC containing Xhol nucleic acid molecule cassette from pCIB10251 into pCIB10252. Each doublet was transferred as an Xbal cassette into the Binary vector pCIB200(KanR). The selectable marker scheme for plastid targeted vectors was: for the fre vector-Right Border-UB10-clp-fre-nos-UB3-selectable marker-nos-Left Border; for PrnA/B vectorsRight Border-UB3-prnA-nos-UB3-prnB-nos-UB3-selectable marker-nos-Left Border and for PrnC/D vectorsRight Border-UB3-prnC-nos-UB3-prnD-nos-UB3-selectable marker-nos-Left.

The plastid targeted prnAB-fre vector is then electroporated into Agrobacterium and used to transform Arabadopsis columbia via the method of N. Bechtold et al. as described above. Seeds are collected, dried down and planted in soil. Transformed plants are identified by spraying the seedlings with the selective agent and selfed to homozygosity.

Similarly, the plastid targeted prnCD/selectable marker vector is introduced into Arabadopsis as described above and the resulting transformants selfed to homozygosity.

The homozygous transformed plants comprising the plastid targeted prnAB-fre/selectable marker construct are then crossed with the homozygous plastid targeted prnCD/selectable marker plants. In another embodiment, the plastid targeted prnCD cassette is transferred into the binary vector comprising the UB10-plastid targeted fre cassette. This vector is known as pNOV524 (SEQ ID NO: 35). The vector pNOV524 is then electroporated into Agrobacterium and used to transform Arabidopsis columbia via the method of N. Bechtold et al. as described above. Both wildtype Arabidopsis and Arabidopsis previously transformed with pCIB10253 (comprising plastid targeted prnA/B) are transformed with pNOV524. Seeds are collected, dried down and planted in soil. Transformed plants are identified by spraying the seedlings with the selective agent and selfed to homozygosity. The resulting progeny are subject to the appropriate selective agent. Plants resistant to this selective agent regime possess fre and prnA, B, C, and D in the hemizygous state. One skilled in the art will recognize the many variations possible in this approach. In all cases, pyrrolnitrin expression is quantified by HPLC or gas chromatography.

### Example 7

Halogenation by PrnC Expressed in Transgenic Plant Leaves Supplied with MDA

Western blot analyses of Columbia lines transformed with pNOV524 construct (comprising the plastid targeted prnC, prnD and fre) are performed following basta selection. Additionally, western blot analyses of Arabidopsis lines transformed with pCIB10253 (comprising plastid targeted prnA and prnB) and subsequently transformed with pNOV 524 are performed following basta selection. Single leaves from each of the lines are homogenized in 1× protein sample buffer, boiled and separated by 10% SDS-PAGE. Subsequently, the membrane is probed for the presence of prnC and prnD proteins with antibodies raised against prnC and prnD, respectively. Arabidopsis lines positive for prnC and prnD expression are identified. The same protein extracts are re-examined for the presence of the flavin reductase (fre) protein using a 10-20% gradient gel and subsequently probing the membrane with antibodies raised against fre. Lines positive for fre expression are identified.

Leaves are taken from an Arabidopsis line positive for plastid-targeted prnC, prnD and fre expression and additionally from an Arabidopsis line that is negative for prnC and prnD by western blot. The leaves are vacuum infiltrated with MDA while submerged in an 5 mM MES (pH5.7); 400 mM Mannitol buffer, and left overnight at room temperature in the dark. Subsequently, the buffer is extracted with ethylacetate, concentrated to dryness and analyzed on the HPLC (as described in the preceding Example 4).

The leaves from plants positive for prnC, prnD and fre convert MDA to APRN (approximately 5%). Conversion is detected within 3 hours of incubation time. Furthermore, approximately 30% of the APRN is converted to pyrrolnitrin. In addition, the negative control, leaves from plants not expressing prnC or prnD, show no conversion of MDA to either APRN or pyrrolnitrin.

The above cited referenced publications are all herein incorporated by reference in their entirety.

What is claimed is:

1. A method of transferring a halogen to a substrate in a regiospecific manner comprising contacting the substrate with a regiospecific halogenase in the presence of an oxidant, a halogen donor, an electron transferase, and a reductant where if the transfer occurs in vivo the electron transferase is encoded by a heterologous nucleic acid molecule.

2. The method of claim 1, further comprising a FAD or FMN component.

3. The method of claim 2, wherein the further component is FAD.

4. The method of claim 2, wherein the electron transferase is an enzyme capable of catalyzing the electron transfer from NADH or NADPH or ferredoxin to FAD.

5. The method of claim 2, wherein the electron transferase is an enzyme capable of catalyzing the electron transfer from NADH or NADPH or ferredoxin to the regiospecific halogenase.

6. The method of claim 2, wherein the electron transferase is a flavin reductase, ferrodoxin NADP reductase, ferredoxin, diaphorase-sufhydryl reductase or NADH-cyt-B5 reductase, NADPH-FMN reductase, NADPH-cyt-p450 reductase or nitrate reductase.

7. The method of claim 6, wherein the electron transferase comprises an amino acid sequence having at least 30% identity to any one of the amino acid sequences according to SEQ ID NOs: 19, 21, 23, 25, 27, 29 or 31.

8. The method of claim 7, wherein the electon transferase comprises an amino acid sequence of any one of SEQ ID NOs: 19, 21, 23, 25, 29 or 31.

9. The method of claim 1, wherein the regiospecific halogenase is prnA, prnC, pyoluteorin halogenases pltA, pltD, and pltM, tetracycline halogenase cts4, hydrolase a, or balhimycin halogenase bha A.

10. The method of claim 9, wherein the regio specific halogenase comprises SEQ ID NO: 1.

11. The method of claim 10, wherein the regio specific halogenase is a polypeptide comprising an amino acid domain according to any one of SEQ ID NOs: 3, 5, 7, 9, 11, 13, 15 or 17.

12. A host cell expressing a heterologous nucleic acid substantially similar to any one of SEQ ID Nos. 18, 10, 22, 24, 26, 28, or 30 and at least one heterologous nucleic acid substantially similar to anyone of SEQ ID NOs: 2, 4, 6, 8, 10, 12, 14 or 16.

13. The host cell of claim 12, wherein the host cell is a bacterial, fungal or plant cell.

14. The host cell of claim 13 wherein the host cell is a microbial cell.

15. The host cell of claim 13, wherein the host cell further expresses nucleic acid sequences encoding prnB and prnD.

16. A method of producing pyrrolnitrin comprising growing the host cell of claim 15.

17. A method of protecting a plant against a pathogen comprising treating the plant with the host cell of claim 15, whereby pyrrolnitrin is produced by the host in amounts that inhibit the pathogen.

18. The method of claim 16, further comprising collecting pyrrolnitrin from the host.

19. A plant comprising a host cell of claim 14.

20. A plant comprising a host cell of claim 15.

21. A method of protecting a plant against a pathogen, comprising growing the plant of claim 20, whereby pyrrolnitrin is produced in the plant in amounts that inhibit the pathogen.

22. A seed of the plant according to claim 20.

23. A method of preventing fungal growth on a crop, comprising growing the plant of claim 21, wherein the plant is a crop plant.

24. A method for improving production of halogenated substrates by a host comprising expressing a heterologous nucleic acid molcule encoding electron transferase in a host wherein the host expresses at least one endogenous polypeptide having regiospecific halogenase activity.


SEQUENCE LISTING (35 sequences)

SEQ ID NO: 1 - protein, 8 aa, Artificial Sequence
  Domain (1). . . (8): Amino acid consensus domain. Xaa1 is G or T; Xaa2 is V,L, T, F, or M; Xaa3 is any amino acid residue; Xaa4 is I, F, M, or L.
XWXWXIPX

SEQ ID NO: 2 - DNA, 1617 bp, Pseudomonas fluorescens
  CDS (1)..(1617)
atgaacaagccgatcaagaatatcgtcatcgtgggcggcggtactgcgggctggatggccgcctcgtacctcgtccgggccctccaacagcaggcgaacattacgctcatcgaatctgcggcgatccctcggatcggcgtgggcgaagcgaccatcccaagtttgcagaaggtgttcttcgatttcctcgggataccggagcgggaatggatgccccaagtgaacggcgcgttcaaggccgcgatcaagttcgtgaattggagaaagtctcccgacccctcgcgcgacgatcacttctaccatttgttcggcaacgtgccgaactgcgacggcgtgccgcttacccactactggctgcgcaagcgcgaacagggcttccagcagccgatggagtacgcgtgctacccgcagcccggggcactcgacggcaagctggcaccgtgcctgtccgacggcacccgccagatgtcccacgcgtggcacttcgacgcgcacctggtggccgacttcttgaagcgctgggccgtcgagcgcggggtgaaccgcgtggtcgatgaggtggtggacgttcgcctgaacaaccgcggctacatctccaacctgctcaccaaggaggggcggacgctggaggcggacctgttcatcgactgctccggcatgcgggggctcctgatcaatcaggcgctgaaggaacccttcatcgacatgtccgactacctgctgtgcgacagcgcggtcgccagcgccgtgcccaacgacgacgcgcgcgatggggtcgagccgtacacctcctcgatcgccatgaactcgggatggacctggaagattccgatgctgggccggttcggcagcggctacgtcttctcgagccatttcacctcgcgcgaccaggccaccgccgacttcctcaaactctggggcctctcggacaatcagccgctcaaccagatcaagttccgggtcgggcgcaacaagcgggcgtgggtcaacaactgcgtctcgatcgggctgtcgtcgtgctttctggagcccctggaatcgacggggatctacttcatctacgcggcgctttaccagctcgtgaagcacttccccgacacctcgttcgacccgcggctgagcgacgctttcaacgccgagatcgtccacatgttcgacgactgccgggatttcgtccaagcgcactatttcaccacgtcgcgcgatgacacgccgttctggctcgcgaaccggcacgacctgcggctctcggacgccatcaaagagaaggttcagcgctacaaggcggggctgccgctgaccaccacgtcgttcgacgattccacgtactacgagaccttcgactacgaattcaagaatttctggttgaacggcaactactactgcatctttgccggcttgggcatgctgcccgaccggtcgctgccgctgttgcagcaccgaccggagtcgatcgagaaagccgaggcgatgttcgccagcatccggcgcgaggccgagcgtctgcgcaccagcctgccgacaaactacgactacctgcggtcgctgcgtgacggcgacgcggggctgtcgcgcggccagcgtgggccgaagctcgcagcgcaggaaagcctgtag

SEQ ID NO: 3 - protein, 538 aa, Pseudomonas fluorescens
MNKPIKNIVIVGGGTAGWMAASYLVRALQQQANITLIESAAIPRIGVGEATIPSLQKVFFDFLGIPEREWMPQVNGAFKAAIKFVNWRKSPDPSRDDHFYHLFGNVPNCDGVPLTHYWLRKREQGFQQPMEYACYPQPGALDGKLAPCLSDGTRQMSHAWHFDAHLVADFLKRWAVERGVNRVVDEVVDVRLNNRGYISNLLTKEGRTLEADLFIDCSGMRGLLINQALKEPFIDMSDYLLCDSAVASAVPNDDARDGVEPYTSSIAMNSGWTWKIPMLGRFGSGYVFSSHFTSRDQATADFLKLWGLSDNQPLNQIKFRVGRNKRAWVNNCVSIGLSSCFLEPLESTGIYFIYAALYQLVKHFPDTSFDPRLSDAFNAEIVHMFDDCRDFVQAHYFTTSRDDTPFWLANRHDLRLSDAIKEKVQRYKAGLPLTTTSFDDSTYYETFDYEFKNFWLNGNYYCIFAGLGMLPDRSLPLLQHRPESIEKAEAMFASIRREAERLRTSLPTNYDYLRSLRDGDAGLSRGQRGPKLAAQESL

SEQ ID NO: 4 - DNA, 1704 bp, Pseudomonas fluorescens
  CDS (1)..(1704)
atgactcagaagagccccgcgaacgaacacgatagcaatcacttcgacgtaatcatcctcggctcgggcatgtccggcacccagatgggggccatcttggccaaacaacagtttcgcgtgctgatcatcgaggagtcgtcgcacccgcggttcacgatcggcgaatcgtcgatccccgagacgtctcttatgaaccgcatcatcgctgatcgctacggcattccggagctcgaccacatcacgtcgttttattcgacgcaacgttacgtcgcgtcgagcacgggcattaagcgcaacttcggcttcgtgttccacaagcccggccaggagcacgacccgaaggagttcacccagtgcgtcattcccgagctgccgtgggggccggagagccattattaccggcaagacgtcgacgcctacttgttgcaagccgccattaaatacggctgcaaggtccaccagaaaactaccgtgaccgaataccacgccgataaagacggcgtcgcggtgaccaccgcccagggcgaacggttcaccggccggtacatgatcgactgcggaggacctcgcgcgccgctcgcgaccaagttcaagctccgcgaagaaccgtgtcgcttcaagacgcactcgcgcagcctctacacgcacatgctcggggtcaagccgttcgacgacatcttcaaggtcaaggggcagcgctggcgctggcacgaggggaccttgcaccacatgttcgagggcggctggctctgggtgattccgttcaacaaccacccgcggtcgaccaacaacctggtgagcgtcggcctgcagctcgacccgcgtgtctacccgaaaaccgacatctccgcacagcaggaattcgatgagttcctcgcgcggttcccgagcatcggggctcagttccgggacgccgtgccggtgcgcgactgggtcaagaccgaccgcctgcaattctcgtcgaacgcctgcgtcggcgaccgctactgcctgatgctgcacgcgaacggcttcatcgacccgctcttctcccgggggctggaaaacaccgcggtgaccatccacgcgctcgcggcgcgcctcatcaaggcgctgcgcgacgacgacttctcccccgagcgcttcgagtacatcgagcgcctgcagcaaaagcttttggaccacaacgacgacttcgtcagctgctgctacacggcgttctcggacttccgcctatgggacgcgttccacaggctgtgggcggtcggcaccatcctcgggcagttccggctcgtgcaggcccacgcgaggttccgcgcgtcgcgcaacgagggcgacctcgatcacctcgacaacgaccctccgtatctcggatacctgtgcgcggacatggaggagtactaccagttgttcaacgacgccaaagccgaggtcgaggccgtgagtgccgggcgcaagccggccgatgaggccgcggcgcggattcacgccctcattgacgaacgagacttcgccaagccgatgttcggcttcgggtactgcatcaccggggacaagccgcagctcaacaactcgaagtacagcctgctgccggcgatgcggctgatgtactggacgcaaacccgcgcgccggcagaggtgaaaaagtacttcgactacaacccgatgttcgcgctgctcaaggcgtacatcacgacccgcatcggcctggcgctgaagaagtag

SEQ ID NO: 5 - protein, 567 aa, Pseudomonas fluorescens
MTQKSPANEHDSNHFDVIILGSGMSGTQMGAILAKQQFRVLIIEESSHPRFTIGESSIPETSLMNRIIADRYGIPELDHITSFYSTQRYVASSTGIKRNFGFVFHKPGQEHDPKEFTQCVIPELPWGPESHYYRQDVDAYLLQAAIKYGCKVHQKTTVTEYHADKDGVAVTTAQGERFTGRYMIDCGGPRAPLATKFKLREEPCRFKTHSRSLYTHMLGVKPFDDIFKVKGQRWRWHEGTLHHMFEGGWLWVIPFNNHPRSTNNLVSVGLQLDPRVYPKTDISAQQEFDEFLARFPSIGAQFRDAVPVRDWVKTDRLQFSSNACVGDRYCLMLHANGFIDPLFSRGLENTAVTIHALAARLIKALRDDDFSPERFEYIERLQQKLLDHNDDFVSCCYTAFSDFRLWDAFHRLWAVGTILGQFRLVQAHARFRASRNEGDLDHLDNDPPYLGYLCADMEEYYQLFNDAKAEVEAVSAGRKPADEAAARIHALIDERDFAKPMFGFGYCITGDKPQLNNSKYSLLPAMRLMYWTQTRAPAEVKKYFDYNPMFALLKAYITTRIGLALKK

SEQ ID NO: 6 - DNA, 1350 bp, Pseudomonas fluorescens
  CDS (1)..(1350)
atgagcgatcatgattatgatgtagtgattatcggtggcgggccggcgggttcgaccatggcctcctacctggcaaaagccggtgtcaaatgcgcggtgttcgaaaaagaactgttcgagcgcgagcatgttggcgagtcgctggtaccggccaccactccggtgctgctggaaatcggggtgatggaaaagatcgagaaagccaacttcccgaagaagttcggcgctgcctggacctcggcagattccggccccgaagacaagatgggcttccaggggctggaccacgatttccgttcggcggaaatcctcttcaacgagcgcaagcaggaaggggtcgatcgcgacttcacgttccacgtcgaccgcggcaagttcgaccgcattcttctggagcacgcaggttcgctgggggccaaggtcttccagggcgtggagatcgctgacgtcgagtttctcagcccgggcaatgtcattgtcaatgccaagctgggcaagcgcagcgtggagatcaaggccaagatggtggtggatgccagcggtcgcaacgtgctgctgggccgccggctgggcttgcgagaaaaggacccggtcttcaaccagttcgcgattcactcctggttcgacaacttcgaccgcaagtcggcgacgcaaagcccggacaaggtcgactacatcttcattcacttcctgccgatgaccaatacctgggtctggcagatcccgatcaccgaaaccattaccagcgtgggcgtggttacgcagaagcagaactacaccaactccgacctcacctatgaagagttcttctgggaagcggtgaagacccgggaaaacctgcatgacgcgctgaaggcatcggagcaggtccgcccgttcaagaaagaggcggactacagctacggcatgaaagaagtctgtggcgacagcttcgtgctgatcggcgatgccgcacggttcgtcgacccgatcttctccagcggcgtcagcgttgcactcaacagtgcgcgcatcgccagcggcgacatcatcgaggcggtgaagaacaacgactttagcaagtccagtttcactcactacgaaggcatgatcaggaatggcatcaagaactggtatgagttcatcacgctctattaccgcctgaacatcctcttcaccgcgttcgttcaagacccacgctaccgcctggacatcctgcaattgctgcaaggggacgtctacagcggcaagcgcctggaagtgctggacaagatgcgcgaaatcatcgctgcggttgaaagcgacccggaacacctctggcacaagtacctgggcgacatgcaggttcctaccgccaaacccgcgttctaa

SEQ ID NO: 7 - protein, 449 aa, Pseudomonas fluorescens
MSDHDYDVVIIGGGPAGSTMASYLAKAGVKCAVFEKELFEREHVGESLVPATTPVLLEIGVMEKIEKANFPKKFGAAWTSADSGPEDKMGFQGLDHDFRSAEILFNERKQEGVDRDFTFHVDRGKFDRILLEHAGSLGAKVFQGVEIADVEFLSPGNVIVNAKLGKRSVEIKAKMVVDASGRNVLLGRRLGLREKDPVFNQFAIHSWFDNFDRKSATQSPDKVDYIFIHFLPMTNTWVWQIPITETITSVGVVTQKQNYTNSDLTYEEFFWEAVKTRENLHDALKASEQVRPFKKEADYSYGMKEVCGDSFVLIGDAARFVDPIFSSGVSVALNSARIASGDIIEAVKNNDFSKSSFTHYEGMIRNGIKNWYEFITLYYRLNILFTAFVQDPRYRLDILQLLQGDVYSGKRLEVLDKMREIIAAVESDPEHLWHKYLGDMQVPTAKPAF

SEQ ID NO: 8 - DNA, 1641 bp, Pseudomonas fluorescens
  CDS (1)..(1641)
gtggttatgaacgatgtgcagtctggcaaggcgccagagcattacgacattctcttggcgggcaacagcatcagcgtgatcatgctcgccgcctgcctggcccggaacaaggtccgggtcggtttgttgcgcaaccggcagatgccccccgaccttaccggtgaggcgacgattccctatacctcgatgattttcgagctgattgccgaccgctatggcgtgccggaaataaagaatatcgcccgcacccgggatatccagcagaaggtgatgccgtcttccggggtcaagaagaacctcgggttcatctatcaccagcgcagccgggcggtggacctgggccaggcgctgcaattcaacgtgccctccgagcatggcgagaaccatctgttcaggcccgatatcgatgcctatctgctggcggcggccatcggttatggcgcgcagctggtggagatcgataacagcccagaggtgctggtcgaggacagcggggtcaaggtagctacggcactggggcgctgggtcactgccgatttcatggttgatggcagccagggcggccaggtgctggcgcggcaggctggcctggtcagccaggcttcgacgcagaagacccggaccctggaattctccactcatatgctcggggtggtgccgttcgatgagtgcgtgcagggcgattttcccggccagtggcatggcggcactctgcatcacgtgttcgatgggggctgggtgggggtcatcccgttcaacaaccatcagcactcgcgcaaccctttggtcagcgtgctggtttcactgcgtgaggacctctgcccgagcatggacggcgaccaggtcctggccggcctgatcgagctgtaccccggcctggggcggcacctgtccggcgcccggcgggtgcgcgagtgggtgctgcgccagccgccccggcaggtctatcgcacggcgctcgaacgccgctgcctgatgttcgacgagggcgccgcgagcaacgatctgttgttctcgcgcaagctgtccaatgctgcggaactggttctggccctggcgcaccggctgatcaaggcggcgcacagcggtgactaccgcagcccggccctgaatgattttgtcctgacccaggacagcatcatcagcttgagtgaccggatcgccttagcggcttatgtgtcgtttcgcgaccccgagttgtggaatgccttcgcccgtgtctggctgctgcagtcgattgccgccaccatcaccgcgcgcaagatcaacgatgcctttgccaaggacctggacccgcgagtgttcgatgaaatcgaccagctcgcagaggacggtttctggatgcctctgtatcgggggtacaaggatattctcaacactacgctgggcctttgtgatgacgtcaaaagcgccaaggtctctgctgcgcacgcggcgagcagcatctttgcggagcttgccaacgccagttttgttccgcctatttttgattttgctaatcctcacgctcgtgtctatcaactgaccaccttgagaaagctcaaggcgctctggtggggcctgatgcaagtgccctcagaggtcggacggctgattttctatcgatccttcagaaaaccttccctgcgcaaggagagttga

SEQ ID NO: 9 - protein, 546 aa, Pseudomonas fluorescens
VVMNDVQSGKAPEHYDILLAGNSISVIMLAACLARNKVRVGLLRNRQMPPDLTGEATIPYTSMIFELIADRYGVPEIKNIARTRDIQQKVMPSSGVKKNLGFIYHQRSRAVDLGQALQFNVPSEHGENHLFRPDIDAYLLAAAIGYGAQLVEIDNSPEVLVEDSGVKVATALGRWVTADFMVDGSQGGQVLARQAGLVSQASTQKTRTLEFSTHMLGVVPFDECVQGDFPGQWHGGTLHHVFDGGWVGVIPFNNHQHSRNPLVSVLVSLREDLCPSMDGDQVLAGLIELYPGLGRHLSGARRVREWVLRQPPRQVYRTALERRCLMFDEGAASNDLLFSRKLSNAAELVLALAHRLIKAAHSGDYRSPALNDFVLTQDSIISLSDRIALAAYVSFRDPELWNAFARVWLLQSIAATITARKINDAFAKDLDPRVFDEIDQLAEDGFWMPLYRGYKDILNTTLGLCDDVKSAKVSAAHAASSIFAELANASFVPPIFDFANPHARVYQLTTLRKLKALWWGLMQVPSEVGRLIFYRSFRKPSLRKES

SEQ ID NO: 10 - DNA, 1510 bp, Pseudomonas fluorescens
  CDS (1)..(1509)
atgaatcagtacgacgtcattatcatcggtagtggtatcgccggcgcgctgaccggcgccgtcctcgcgaagtccgggctgaacgttctgatcctcgactcggcccagcacccacgattctccgtcggcgaagcggcgacaccggaaagcggttttctgctgcgtttgctctcaaagcgcttcgacatccctgaaatcgcctacctctcgcaccccgacaagatcatccagcacgtcggttcgagcgcctgcgggatcaagctgggcttcagttttgcctggcatcaagagaacgcgccgtcgtcccccgaccaccttgtggccccgccgctgaaggtgccggaagcccatcttttccggcaggacatcgactatttcgccctgatgattgccctgaaacacggcgccgaatccagacagaacatcaagatcgagtcgatcagcctcaacgacgacggggtcgaggtggcattgtccaacgccgcccccgtcaaggccgcgttcatcattgacgctgctgcccagggctctccgctttcccgccaactgggcttgcgcaccaccgaagggctggcgaccgacacctgctcattcttcacccacatgctcaatgtgaagagctacgaagatgccctggctccgttgtcccgcactcgttcccccatcgaactgttcaagagcaccttgcaccacatcttcgaagagggctggttgtgggtcatccccttcaacaaccacccgcagggcaccaatcagttgtgcagcatcggcttccagttcaacaacgccaagtaccgtcccaccgaggcgccggagatcgagtttcgcaaactgctgaaaaagtacccggccatcggcgaacacttcaaggatgcggtcaatgcccgggagtggatctacgcgccgcgcatcaactaccgcagcgtgcaaaatgtcggggatcgcttctgcctgctgccgcaagccacagggtttatcgacccgctgttctccagggggttgatcaccaccttcgagtccatcctcaggctggcccccaaggtgctggacgccgcccgcagcaaccgctggcaacgggaacagttcatcgaagtcgagcgccattgcctgaacgcggtggcgaccaatgaccagttggtctcctgctcctatgaagccttcagcgactttcacctgtggaacgtgtggcatcgggtctggctcagcggctccaacctgggcagtgcctttctgcaaaagctgctgcacgacctggaacacagtggcgacgcccgccagttcgatgcagcgcttgaggcggtgcgcttccctggctgcctgtccctggactcgcccgcctacgaaagcctgttcaggcagtcgtgccaggtcatgcaacaggccagggagcaagccaggccggtggccgaaaccgccaacgcgctgcatgagctgatcaaggagcacgaagccgagttgttgcccctgggctattcacggatatccaatcgtttcatcctcaaagtctgaa

SEQ ID NO: 11 - protein, 502 aa, Pseudomonas fluorescens
MNQYDVIIIGSGIAGALTGAVLAKSGLNVLILDSAQHPRFSVGEAATPESGFLLRLLSKRFDIPEIAYLSHPDKIIQHVGSSACGIKLGFSFAWHQENAPSSPDHLVAPPLKVPEAHLFRQDIDYFALMIALKHGAESRQNIKIESISLNDDGVEVALSNAAPVKAAFIIDAAAQGSPLSRQLGLRTTEGLATDTCSFFTHMLNVKSYEDALAPLSRTRSPIELFKSTLHHIFEEGWLWVIPFNNHPQGTNQLCSIGFQFNNAKYRPTEAPEIEFRKLLKKYPAIGEHFKDAVNAREWIYAPRINYRSVQNVGDRFCLLPQATGFIDPLFSRGLITTFESILRLAPKVLDAARSNRWQREQFIEVERHCLNAVATNDQLVSCSYEAFSDFHLWNVWHRVWLSGSNLGSAFLQKLLHDLEHSGDARQFDAALEAVRFPGCLSLDSPAYESLFRQSCQVMQQAREQARPVAETANALHELIKEHEAELLPLGYSRISNRFILKV

SEQ ID NO: 12 - DNA, 1476 bp, Amycolatopsis orientalis
  CDS (1)..(1476)
atgtcggtcgaagatttcgatgttgtggtggcgggcggcgggccgggtggttcgacggtggccaccctggtggcgatgcagggacaccgggtcctgctgctggagaaagaggtcttcccccggtaccagatcggtgagtcgctgctgcccgccacggtgcacggggtctgccggatgctcggcgtcgcggacgagctggcgaattccgggttcccgatcaaacgcggcggcacgttccgctggggcgcccgtccggagccgtggacgttccacttcgggatctcggccaagatggcgggctcgacgtcgcacgcctatcaggtcgagcgggcgaagttcgacgacatcctgctgaagaacgccaagagcaagggcgtcgtcgtgcgggaaggctgctcggtcaacgacgtcgtggaggacggcgagcgggtcaccggcgcgcgctacaccgacgcggacggcaacgcgcacgaagtctcggcccggttcgtgatcgacgcgtcgggcaacaagagccggctctacacgaaggtcaacggttcgcggaactactcggagttcttccgcagcctcgcgctgttcggctatttcgagggtggcaaacggctgcccgagccggtgtcgggcaacatcctgagcgtcgccttcgacagcggctggttctggtacatccccctgagcgacacgctgaccagcgtcggcgcggtcgtgcgccgggaggacgccgacaagatccagggcgaccgcgagaaggccctcaacaccttgatcgccgaatgcccgctgatctcggagtacctctcgaacgcgaccagggtgaccaccggcaggtacggcgaactgcgggtgcgcaaggactactcgtaccagcaggacagctactggcggcccgggatggtcctggtcggcgacgccgcgtgcttcgtggacccggtgttctcctccggggtgcacctggcgacctacagcgcgctgctcgcggcccggtcgatcaacagcgtcctcgcgggcgacctcgacgagaagaccgcgctgaacgagttcgaggcgcgctatcgccgcgagtacggcgtcttctacgagttcctcgtctccttctatcagatgaacgtcaacgaggaatcgtatttctggcaggccaagaaggtcacgcagaaccagagcaccgacatcgagtcgttcgtcgagctgatcggcggggtgtcgtccggcgagaccgcgctgacggccgccgaccggatcgccgcgaacagtgccgaattcgccgccgccgtcgacaagatggcgacgggcgacggcgacgacatggtgccgatgttcaagtcgaccgtggtcaagcaggcgatgcaggaggcgggccaggtccagatgaaggcgctgctcggcgaggacgccgaacccgagctgccgctgttccccggcggcctggtgacttcgcccgacgggatgaagtggctgccgcaccacccggcctga

SEQ ID NO: 13 - protein, 491 aa, Amycolatopsis orientalis
MSVEDFDVVVAGGGPGGSTVATLVAMQGHRVLLLEKEVFPRYQIGESLLPATVHGVCRMLGVADELANSGFPIKRGGTFRWGARPEPWTFHFGISAKMAGSTSHAYQVERAKFDDILLKNAKSKGVVVREGCSVNDVVEDGERVTGARYTDADGNAHEVSARFVIDASGNKSRLYTKVNGSRNYSEFFRSLALFGYFEGGKRLPEPVSGNILSVAFDSGWFWYIPLSDTLTSVGAVVRREDADKIQGDREKALNTLIAECPLISEYLSNATRVTTGRYGELRVRKDYSYQQDSYWRPGMVLVGDAACFVDPVFSSGVHLATYSALLAARSINSVLAGDLDEKTALNEFEARYRREYGVFYEFLVSFYQMNVNEESYFWQAKKVTQNQSTDIESFVELIGGVSSGETALTAADRIAANSAEFAAAVDKMATGDGDDMVPMFKSTVVKQAMQEAGQVQMKALLGEDAEPELPLFPGGLVTSPDGMKWLPHHPA

SEQ ID NO: 14 - DNA, 1359 bp, Streptomyces aureofaciens
  CDS (1)..(1359)
atgttccaccgggacggcgaggagccggacccgaacgagaccagccagttccgcatcccctcgatcgtcggcaacgcggcccacttcttccgccaggacaccgactcctacatgttccacgccgcggtgcgctacggctgcgacgcccggcagtactaccgggtggagaacatcgagttcgacgacggcggggtgaccgtctccggcgcggacggcagcaccgtccgggcccgctacctggtcgacgccagcggcttccgctcgccgctggcacggcagttggggttgcgggaggagccgagccggctcaagcaccacgcccgctcgatcttcacccacatggtcggagtggacgcgatcgacgaccacgtggacatgccggccgagcttcgcccgccggtgccgtggaacgacgggacgatgcaccacatcttcgagcgcggctggatgtggatcatcccgttcaacaaccaccccggggccaccaacccgctgtgcagcgtcggcatccagctcgacgagcgccgctaccccgcccggccggacctgacgcccgaggaggagttccggtcccacgtggaccgcttcccggcggtgcagcggcagttgaagggcgcccgcagcgtgcgcgagtgggtgcgaacggaccgcatgcagtactcctcgagccggacggtcggcgagcgctggtgcctgatgtcgcacgcggccggcttcatcgacccgctcttccttcgcggcctgtccaacacctgcgagatcatcaacgcgctgtcctggcggctgatggccgcgctgcgcgaggacgacttcgcggtcgagcgcttcgcctacgtggaggaactggagcagggcctgctggactggaacgacaagctggtcaacaactccttcatctccttctcgcactacccgctgtggaactcggcgttccggatctgggcctcggccagcgtgatcggcggcaagcgcatcctcaacgcactgaccaggaccaaggagaccggcgacgacagccactgccaggcgctggacgacaacccgtacccgggcctgtggtgtccgctggacttctacaaggaggccttcgacgagctcaccgagctgtgcgaggccgtggacgccgggcacaccacggccgaggaggccgcgcggctgctggagcagcgggtccgcgagtcggactggatgctgccggccctgggcttcaacgaccccgacacccaccacatcaacccgacggcggacaagatgatccggatcgcggagtgggccaccggtcaccaccgcccggagatccgtgagctgctggccgccagcgccgaggaggtcagggcggcgatgcgggtcaagccgtaa

SEQ ID NO: 15 - protein, 452 aa, Streptomyces aureofaciens
MFHRDGEEPDPNETSQFRIPSIVGNAAHFFRQDTDSYMFHAAVRYGCDARQYYRVENIEFDDGGVTVSGADGSTVRARYLVDASGFRSPLARQLGLREEPSRLKHHARSIFTHMVGVDAIDDHVDMPAELRPPVPWNDGTMHHIFERGWMWIIPFNNHPGATNPLCSVGIQLDERRYPARPDLTPEEEFRSHVDRFPAVQRQLKGARSVREWVRTDRMQYSSSRTVGERWCLMSHAAGFIDPLFLRGLSNTCEIINALSWRLMAALREDDFAVERFAYVEELEQGLLDWNDKLVNNSFISFSHYPLWNSAFRIWASASVIGGKRILNALTRTKETGDDSHCQALDDNPYPGLWCPLDFYKEAFDELTELCEAVDAGHTTAEEAARLLEQRVRESDWMLPALGFNDPDTHHINPTADKMIRIAEWATGHHRPEIRELLAASAEEVRAAMRVKP

SEQ ID NO: 16 - DNA, 1476 bp, Amycolatopsis mediterranei
  CDS (1)..(1476)
atgtcggtcgaagacttcgacgtggtggtggcgggcggcgggccgggtggttcgacggtggccacgctggtggccatgcagggacaccgggtgctgctgctggagaaagaggttttcccgcggtatcagatcggtgagtcgctgctgcccgccacggtgcacggcgtgtgccggatgctcggcatctccgacgagctggccaatgccgggttcccgatcaagcgcggcggcacgttccgctggggcgcccggccggagccgtggacgttccacttcggcatctcggccaagatggccggctcgacgtcgcacgcctaccaggtcgagcgggcgcggttcgacgagatgctgctgaacaacgccaagcgcaagggcgtggtcgtgcgggaggggtgcgcggtcaccgatgtggtggaagacggcgagcgggtcaccggtgcgcggtacaccgatcccgacggcaccgagcgggaagtgtcggcgcggttcgtgatcgacgcgtcgggcaacaagagccggctctacaccaaggtcggcggttcgcggaactattcggagttcttccgcagcctcgcgctgttcggttacttcgagggtggcaagcggctgcccgagccggtctccgggaacatcctgagtgtggccttcgacagcggctggttctggtacatcccgctgagcgacacgctgaccagcgtcggcgcggtggtgcgccgggaggacgccgagaagatccagggtgaccgggagaaggccctcaacacgctgatcgccgagtgcccgctgatctcggaatacctcgcggacgcgacccgggtgacgaccggccggtacggggaactgcgcgtccgcaaggactactcctaccagcaggagacctactggcggccgggcatgatcctggtcggcgacgccgcgtgtttcgtggacccggtgttctcctccggtgtgcacctggcgacctacagcgcgctgctcgcggcccggtcgatcaacagcgtcctcgccggcgacctggacgagaagaccgcgctgaacgagttcgagctgcggtatcgccgtgagtacggcgtgttctacgagttcctcgtgtccttctaccagatgaacgtgaacgaggagtcgtacttctggcaggccaagaaggtcacgcagaaccagagcaccgacgtcgagtcgttcgtcgagctgatcggcggagtgtcgtccggggagaccgcgctgacggccgccgaccgcatcgccgcgcgcagtgccgagttcgccgcggcggtggacgagatggcgggcggggacggcgacaacatggtgccgatgttcaagtcgacggtggtccagcaggcgatgcaggaagcgggccaggtgcagatgaaggcgctgctcggcgaggacgccgaacccgagctgcccctgttccccggtggcctggtgacctcgcccgaacggatgaagtggctgcctcaccaccctgcgtga

SEQ ID NO: 17 - protein, 491 aa, Amycolatopsis mediterranei
MSVEDFDVVVAGGGPGGSTVATLVAMQGHRVLLLEKEVFPRYQIGESLLPATVHGVCRMLGISDELANAGFPIKRGGTFRWGARPEPWTFHFGISAKMAGSTSHAYQVERARFDEMLLNNAKRKGVVVREGCAVTDVVEDGERVTGARYTDPDGTEREVSARFVIDASGNKSRLYTKVGGSRNYSEFFRSLALFGYFEGGKRLPEPVSGNILSVAFDSGWFWYIPLSDTLTSVGAVVRREDAEKIQGDREKALNTLIAECPLISEYLADATRVTTGRYGELRVRKDYSYQQETYWRPGMILVGDAACFVDPVFSSGVHLATYSALLAARSINSVLAGDLDEKTALNEFELRYRREYGVFYEFLVSFYQMNVNEESYFWQAKKVTQNQSTDVESFVELIGGVSSGETALTAADRIAARSAEFAAAVDEMAGGDGDNMVPMFKSTVVQQAMQEAGQVQMKALLGEDAEPELPLFPGGLVTSPERMKWLPHHPA

SEQ ID NO: 18 - DNA, 702 bp, Escherichia coli
  CDS (1)..(702)
atgacaaccttaagctgtaaagtgacctcggtagaagctatcacggataccgtatatcgtgtccgcatcgtgccagacgcggccttttcttttcgtgctggtcagtatttgatggtagtgatggatgagcgcgacaaacgtccgttctcaatggcttcgacgccggatgaaaaagggtttatcgagctgcatattggcgcttctgaaatcaacctttacgcgaaagcagtcatggaccgcatcctcaaagatcatcaaatcgtggtcgacattccccacggagaagcgtggctgcgcgatgatgaagagcgtccgatgattttgattgcgggcggcaccgggttctcttatgcccgctcgattttgctgacagcgttggcgcgtaacccaaaccgtgatatcaccatttactggggcgggcgtgaagagcagcatctgtatgatctctgcgagcttgaggcgctttcgttgaagcatcctggtctgcaagtggtgccggtggttgaacaaccggaagcgggctggcgtgggcgtactggcaccgtgttaacggcggtattgcaggatcacggtacgctggcagagcatgatatctatattgccggacgttttgagatggcgaaaattgcccgcgatctgttttgcagtgagcgtaatgcgcgggaagatcgcctgtttggcgatgcgtttgcatttatctga

SEQ ID NO: 19 - protein, 233 aa, Escherichia coli
MTTLSCKVTSVEAITDTVYRVRIVPDAAFSFRAGQYLMVVMDERDKRPFSMASTPDEKGFIELHIGASEINLYAKAVMDRILKDHQIVVDIPHGEAWLRDDEERPMILIAGGTGFSYARSILLTALARNPNRDITIYWGGREEQHLYDLCELEALSLKHPGLQVVPVVEQPEAGWRGRTGTVLTAVLQDHGTLAEHDIYIAGRFEMAKIARDLFCSERNAREDRLFGDAFAFI

SEQ ID NO: 20 - DNA, 906 bp, Rattus norvegicus
  CDS (1)..(906)
atgggggcccagctgagcacgttgagccgagtggtactctccccggtctggttcgtctacagcctcttcatgaagctgtttcagcgctcctcaccggccatcaccctcgagaaccccgacatcaagtaccctctgcggctcatcgacaaggagattatcagccatgacactcggcgcttccgatttgcactcccttcgccccagcacatcctgggccttcctatcggccagcacatctacctctccaccaggatcgatggcaacttggtcattcgtccctacacccctgtgtctagtgatgatgacaagggccttgtggacttggtggtcaaggtttacttcaaggacacgcatcccaagtttccagctggagggaaaatgtctcagtacctggaaaacatgaatattggagacaccattgaattccggggccccaatgggctactggtctaccagggcaaagggaagttcgccatccgtgcagacaagaagtccaaccctgttgtcaggacggtgaagtctgtaggcatgattgcaggagggacaggcatcaccccaatgctgcaggtgatccgagccgtcttgaaggacccgaacgaccacactgtgtgctatctgctcttcgccaaccagtccgagaaagacatcctgctgcggcctgagctggaggaactgaggaacgaacattcttctcgcttcaagctctggtacacagtggacaaagcccccgatgcctgggactatagccaaggcttcgtgaatgaggagatgatcagggaccatcttccacctcctggggaggagacactgatactgatgtgtggacccccaccgatgatccagtttgcctgtttgccaaacctggagcgtgtgggccatcccaaggagcgatgcttcaccttctga

SEQ ID NO: 21 - protein, 301 aa, Rattus norvegicus
MGAQLSTLSRVVLSPVWFVYSLFMKLFQRSSPAITLENPDIKYPLRLIDKEIISHDTRRFRFALPSPQHILGLPIGQHIYLSTRIDGNLVIRPYTPVSSDDDKGLVDLVVKVYFKDTHPKFPAGGKMSQYLENMNIGDTIEFRGPNGLLVYQGKGKFAIRADKKSNPVVRTVKSVGMIAGGTGITPMLQVIRAVLKDPNDHTVCYLLFANQSEKDILLRPELEELRNEHSSRFKLWYTVDKAPDAWDYSQGFVNEEMIRDHLPPPGEETLILMCGPPPMIQFACLPNLERVGHPKERCFTF

SEQ ID NO: 22 - DNA, 2049 bp, Oryctolagus cuniculus
  CDS (1)..(2049)
ctgatcaacatggcggactcccacggggacaccggcgccaccatgcctgaagcggcggcccaggaggcgtcggtcttcagcatgacggacgtggttctgttctcgctcatcgtggggctgatcacctactggttcctcttcagaaagaaaaaggaggaagtgcccgagttcaccaagatccaggccccgacgtcgtcgtcagtgaaggagagcagcttcgtggagaagatgaagaagacgggccggaacatcgtggtcttctacggctcccagacgggcaccgccgaggagtttgccaaccgcctgtccaaggatgcccaccgctacgggatgcggggcatggccgccgaccccgaggagtacgacctggccgacctgagcagcctgcccgagatcaacaacgccctggccgtcttctgcatggccacctacggtgagggggaccccaccgacaacgcccaggacttctacgactggctgcaggagaccgacgtggacctctcgggggtcaagtacgcggtgtttggcctcgggaacaagacctacgagcacttcaacgccatgggcaagtacgtggaccagcggctggagcagcttggcgcccagcgcatcttcgagctgggcatgggcgacgatgatgcaaacctggaggaggacttcatcacgtggcgggagcagttctggccggcggtgtgcgagcacttcggtgtggaggccacaggagaggagtccagcattcggcagtacgagctcgtgttgcacacagacatcgacgtggccaaggtgtaccagggcgagatgggccgcctcaagagctacgagaaccagaaaccccccttcgatgccaagaatcccttcctggccacggtcaccaccaaccggaagctgaaccagggcaccgagcgccacctcatgcacctggagctggacatctcggactccaagatcaggtatgagtctggggaccacgtggctgtgtatccggccaacgactctgccctcgtcaaccagctgggggagatcctgggtgccgacctggacgtcgtcatgtccctgaacaacctcgatgaggagtccaacaagaagcacccattcccctgccccacttcctaccgcacggccctcacctactacctggacatcaccaacccgccgcgcaccaacgtgctctacgagctggcccagtacgccgccgaccccgctgagcaggagcagctgcgcaagatggcctcatcctcgggcgagggcaaggagctgtacctgagctgggtggtagaggcgcggaggcacatcctggccatcctccaagactacccgtccctgcggccgcccatcgaccacctgtgtgagctgctgccccggctgcaggcgcgctactactccatcgcctcctcctccaaggtccaccccaactccgtgcacatctgcgccgtggccgtggagtacgagaccaaggccggccgcctcaacaaaggcgtggccaccagctggctgcgggccaaggagccggccggggagaatggcggccgtgccctggtgcccatgttcgtgcgcaagtcccagttccgcctgcccttcaaggccaccacgccggtcatcatggtgggccccggcaccggcgtggcccccttcatcggctttatccaggagcgggcctggctgcggcagcagggcaaggaagtgggcgagacgctgctgtactacggctgccggcgcgcggccgaggactacctgtaccgcgaggagctcgccggcttccaaaaggacggcacgctcagccagctcaacgtggccttctcccgcgagcaggcccagaaggtctacgtgcagcacttgctgaggagggacaaggagcacctgtggcggctcatccacgaggggggcgcccacatctacgtgtgcggggacgctcggaacatggccagggacgtgcagaacaccttctacgacatcgtggccgagctgggggccatggagcacgcgcaggccgtggactacgtgaagaagctcatgaccaagggccgctactccctggacgtgtggagctag

SEQ ID NO: 23 - protein, 682 aa, Oryctolagus cuniculus
LINMADSHGDTGATMPEAAAQEASVFSMTDVVLFSLIVGLITYWFLFRKKKEEVPEFTKIQAPTSSSVKESSFVEKMKKTGRNIVVFYGSQTGTAEEFANRLSKDAHRYGMRGMAADPEEYDLADLSSLPEINNALAVFCMATYGEGDPTDNAQDFYDWLQETDVDLSGVKYAVFGLGNKTYEHFNAMGKYVDQRLEQLGAQRIFELGMGDDDANLEEDFITWREQFWPAVCEHFGVEATGEESSIRQYELVLHTDIDVAKVYQGEMGRLKSYENQKPPFDAKNPFLATVTTNRKLNQGTERHLMHLELDISDSKIRYESGDHVAVYPANDSALVNQLGEILGADLDVVMSLNNLDEESNKKHPFPCPTSYRTALTYYLDITNPPRTNVLYELAQYAADPAEQEQLRKMASSSGEGKELYLSWVVEARRHILAILQDYPSLRPPIDHLCELLPRLQARYYSIASSSKVHPNSVHICAVAVEYETKAGRLNKGVATSWLRAKEPAGENGGRALVPMFVRKSQFRLPFKATTPVIMVGPGTGVAPFIGFIQERAWLRQQGKEVGETLLYYGCRRAAEDYLYREELAGFQKDGTLSQLNVAFSREQAQKVYVQHLLRRDKEHLWRLIHEGGAHIYVCGDARNMARDVQNTFYDIVAELGAMEHAQAVDYVKKLMTKGRYSLDVWS

SEQ ID NO: 24 - DNA, 444 bp, Spinacia oleracea
  CDS (1)..(444)
atggcagcaaccaccacaacaatgatgggcatggccaccacctttgtcccaaaaccccaagcaccaccaatgatggcggcgcttccatccaacaccggccgctctttgttcggactcaagaccggtagccgtggcggaaggatgacaatggctgcctacaaggtaaccttggtaacacccaccggtaacgtagagtttcaatgcccagacgatgtttacatcttggatgctgctgaagaagaaggcattgacttgccttactcatgcagagctgggtcgtgctcttcatgcgccggaaagcttaagacaggtagtcttaaccaagatgatcagagttttttggatgacgatcagatcgatgaaggatgggttcttacctgtgctgcttaccctgttagtgatgttactattgagacccacaaggaagaggagcttactgcctaa

SEQ ID NO: 25 - protein, 147 aa, Spinacia oleracea
MAATTTTMMGMATTFVPKPQAPPMMAALPSNTGRSLFGLKTGSRGGRMTMAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTA

SEQ ID NO: 26 - DNA, 711 bp, Vibrio fischeri
  CDS (1)..(711)
atgccaatcaattgcaaagtaaagtctatcgagccattggcttgtaatacttttcgaattttacttcacccagaacagcctgttgcttttaaagcaggccaatacctaacggttgttatgggtgaaaaagacaaacgcccattctcaatcgcaagtagtccttgtcgccacgaaggtgaaattgagttacatattggtgccgcagagcacaatgcttatgccggagaagtggttgaatcaatgaaatcggcactagaaacgggtggtgatattttaattgatgcgcctcatggtgaagcgtggatccgtgaagacagcgatcgttcaatgttattgattgctggcggtacaggttttagttacgtacgttcaattcttgatcactgtattagccaacagattcaaaaaccaatttacctatactggggtggtcgtgatgaatgccaactgtatgcaaaagcagaattagagagcattgctcaagcgcatagccatattacgtttgtgccagtggttgagaaaagtgaaggctggacaggtaaaacgggtaatgtgttagaagcggtaaaagccgattttaactcactagcagatatggatatttacatcgcaggtcgctttgaaatggctggtgcagcacgtgagcagttcaccactgaaaaacaagcgaagaaagagcagctgtttggtgatgcattcgcatttatctaa

SEQ ID NO: 27 - protein, 236 aa, Vibrio fischeri
MPINCKVKSIEPLACNTFRILLHPEQPVAFKAGQYLTVVMGEKDKRPFSIASSPCRHEGEIELHIGAAEHNAYAGEVVESMKSALETGGDILIDAPHGEAWIREDSDRSMLLIAGGTGFSYVRSILDHCISQQIQKPIYLYWGGRDECQLYAKAELESIAQAHSHITFVPVVEKSEGWTGKTGNVLEAVKADFNSLADMDIYIAGRFEMAGAAREQFTTEKQAKKEQLFGDAFAFI

SEQ ID NO: 28 - DNA, 1110 bp, Spinacia oleracea
  CDS (1)..(1110)
atgaccaccgctgtcaccgccgctgtttctttcccctctaccaaaaccacctctctctccgcccgaagctcctccgtcatttcccctgacaaaatcagctacaaaaaggttcctttgtactacaggaatgtatctgcaactgggaaaatgggacccatcagggcccagatcgcctctgatgtggaggcacctccacctgctcctgctaaggtagagaaacattcaaagaaaatggaggaaggcattacagtgaacaagtttaagcctaagaccccttacgttggaagatgtcttcttaacaccaaaattactggggatgatgcacccggagagacctggcacatggttttttcccatgaaggagagatcccttacagagaagggcaatccgttggggttattccagatggggaagacaagaatggaaagccccataagttgagattgtactcgatcgccagcagtgctcttggtgattttggtgatgctaaatctgtttcgttgtgtgtaaaacgactcatctacaccaatgacgctggagagacgatcaagggagtctgctccaacttcttgtgtgacttgaaacccggtgctgaagtgaagttaacaggaccagttggaaaggagatgctcatgcccaaagaccctaacgcgacaattatcatgcttggaactggaacggggattgctcctttccgttcattcttgtggaagatgttcttcgaaaagcatgatgattacaagtttaacggcttggcttggcttttcttgggtgtacccacaagcagttctcttctctacaaagaggaatttgagaagatgaaggaaaaggctccagacaacttcaggctggattttgcagtgagcagagagcaaactaacgagaaaggggagaagatgtacattcaaacccgaatggcacaatacgcagttgagctatgggaaatgttgaagaaagataatacttatgtctacatgtgtggtctcaagggaatggaaaagggaattgacgacattatggtttcattggctgctgcagaaggcattgattggattgaatacaagaggcagttgaagaaggcagaacaatggaacgttgaagtctactaa

SEQ ID NO: 29 - protein, 369 aa, Spinacia oleracea
MTTAVTAAVSFPSTKTTSLSARSSSVISPDKISYKKVPLYYRNVSATGKMGPIRAQIASDVEAPPPAPAKVEKHSKKMEEGITVNKFKPKTPYVGRCLLNTKITGDDAPGETWHMVFSHEGEIPYREGQSVGVIPDGEDKNGKPHKLRLYSIASSALGDFGDAKSVSLCVKRLIYTNDAGETIKGVCSNFLCDLKPGAEVKLTGPVGKEMLMPKDPNATIIMLGTGTGIAPFRSFLWKMFFEKHDDYKFNGLAWLFLGVPTSSSLLYKEEFEKMKEKAPDNFRLDFAVSREQTNEKGEKMYIQTRMAQYAVELWEMLKKDNTYVYMCGLKGMEKGIDDIMVSLAAAEGIDWIEYKRQLKKAEQWNVEVY

SEQ ID NO: 30 - DNA, 2580 bp, Aspergillus parasiticus
  CDS (1)..(2580)
atggcaaccatcacggaggttcggacggatgcgctcgtcccaactgacctcgtccttaagacaggtcagatcaaaattcaaagcgaagagatctcgacgaaagacctgtccgatatccctctgccaccaccatcaaaacggccgacagaagtgctgagcgtagataaaggaaccccagatagccatgttccgcgtgatcctcgactcatcagattaacgggtgttcatccgtttaacgttgagccacctcttacagatctgtataaagaagggtttttaacatcgccggagctcttctatgttcgaaatcatggcccagtccctcatgtcaaggatgaagatatccctcactgggaaattactatcgaaggactggtagagaagcctttggtactaaacttccgacaagtgttgcagcagtacgaccaaataacggcgcccatcaccctcgtatgtgcaggcaatcgacgcaaagagcaaaacattgtacgtaaaacgaaaggtttttcttggggatccgcgggactatcgactgccctcttcactggcccattgctggcggatatcctccgcagtggcaaacccctgcgtcaagcgaaatacgtctgtatggaaggagcggataagctgcccaatggtcactacggcacactcattaaattgaactgggccctagaccccaacagggggatcatgcttgcacataaaatgaacggggagtctcttcgcccagatcatggtcgtccgctgagggccgtcgtgcccggtcaaataggaggacgaagtgtcaagtggctgaagaggctgatcttgaccgatgcaccaagcgataactggtaccatatcaatgacaaccgcgtcttaccaacaatggtctcgcccgatatggcatcaaataaccgaaattggtggcacgatgagcgggatgcgatttatgacctaaacaccaactccgccgttggatatcctcaaaacaatgaggtcttaaatatcctggaggccagggccgtcatatactgtcagaggatagcttacgctggtgggggccgtagggttaccagggtagaaatatccctagacaaaggcaaatcttggagattggcggatatcgaatatgccgaagacaagtatcgtgatttcgaaggcgagctttttggaggcaaagtagatatgtactggcgcgaaacttgcttctgctggtgtttttggtctctaagcatcgccatcccagagcttgagaacagtgatgccatccttgtaagagccatggatgaagcattgggcgtgcagcctcgcgatatgtactggtccgttctcggaatgatgaacaacccttggttccgggttacaattacgaaggaaaacgggaacttgagattcgagcaccctacccaccctagtatgcctacaggatggatggaacgcgtcaaaaaagctgggggtgacccgacgaatggtaactggggagaaagacacgaaggagaggagccgacggagccggagcccgtgcaagacattaatatgaagaaagacgggccaagccgaacgattagttttgaagaattcaaggagaattcctgtgatgagaagccatggttcatcgtgaatggagaagtgtatgatggtcaagcatttcttgaaggccaccctggcggacggcagagtattatctcctctgccggtcaggacgtctccgaggaattccttgctattcatagcgagacggcaaaggcgatgatgcctgagtaccatattggaacgacggatccggaaggcttgatagcactcaaggatgatgcatcatcctccaccgatgaaattcgcccagtgttcctccaatcacggtcttggacaaaggcaacattgaaagaaaggaaagacatatcatgggatacacgaatatttagtttcaaattggaacacgaagatcaaacattgggtttaccagtcggccagcatcttatgatcaaagtcctcgacagatcatccaacaacgaagccatcatccgctcatacaccccgatttctgaaaccagccaaaaagggactgtggacttgctggttaaagtatactttgcaacagccacctcggcaggcggcaagatgacgatggccctggataggctgccattgggctccgtggtcgaatatcttggaaatggacgagttctcataagtggcaaggagcgccatgttcggtcgtttaagatgatttgtggaggaaccggtatcacaccgatcttgcaggtcttgcgcgccgtggttcaggaccatcaagatcctacctcttgtgtagtcctcaatggaaacagacaggaggaagatatccttcgccgggctgagctcgacggcttcatggcgtccgacagcagaaggtgtaatataatacacactctatccaaagcgccggactcatggactggccgccgaggacgcatatccgaagagctcctaaaggagtacgcggctccagaagatgagagtatggtactgatttgtggtccgccagccatggaagaatcggctcggaggatactgttggcggaaggatggaaagaatcagaccttcactttttctga

SEQ ID NO: 31 - protein, 859 aa, Aspergillus parasiticus
MATITEVRTDALVPTDLVLKTGQIKIQSEEISTKDLSDIPLPPPSKRPTEVLSVDKGTPDSHVPRDPRLIRLTGVHPFNVEPPLTDLYKEGFLTSPELFYVRNHGPVPHVKDEDIPHWEITIEGLVEKPLVLNFRQVLQQYDQITAPITLVCAGNRRKEQNIVRKTKGFSWGSAGLSTALFTGPLLADILRSGKPLRQAKYVCMEGADKLPNGHYGTLIKLNWALDPNRGIMLAHKMNGESLRPDHGRPLRAVVPGQIGGRSVKWLKRLILTDAPSDNWYHINDNRVLPTMVSPDMASNNRNWWHDERDAIYDLNTNSAVGYPQNNEVLNILEARAVIYCQRIAYAGGGRRVTRVEISLDKGKSWRLADIEYAEDKYRDFEGELFGGKVDMYWRETCFCWCFWSLSIAIPELENSDAILVRAMDEALGVQPRDMYWSVLGMMNNPWFRVTITKENGNLRFEHPTHPSMPTGWMERVKKAGGDPTNGNWGERHEGEEPTEPEPVQDINMKKDGPSRTISFEEFKENSCDEKPWFIVNGEVYDGQAFLEGHPGGRQSIISSAGQDVSEEFLAIHSETAKAMMPEYHIGTTDPEGLIALKDDASSSTDEIRPVFLQSRSWTKATLKERKDISWDTRIFSFKLEHEDQTLGLPVGQHLMIKVLDRSSNNEAIIRSYTPISETSQKGTVDLLVKVYFATATSAGGKMTMALDRLPLGSVVEYLGNGRVLISGKERHVRSFKMICGGTGITPILQVLRAVVQDHQDPTSCVVLNGNRQEEDILRRAELDGFMASDSRRCNIIHTLSKAPDSWTGRRGRISEELLKEYAAPEDESMVLICGPPAMEESARRILLAEGWKESDLHFF

SEQ ID NO: 32 - DNA, 37 bp, Artificial Sequence
  misc_feature (1)..(37): primer
gcgcgaattcatgacaaccttaagctgtaaagtgacc

SEQ ID NO: 33 - DNA, 34 bp, Artificial Sequence
  misc_feature (1)..(34): primer
gcgcctgcagtcagataaatgcaaacgcatcgcc

SEQ ID NO: 34 - DNA, 26019 bp, synthetic construct
  misc_feature (1)..(26019): prnA,B,C,D and fre transformation construct
tggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctatcgttctagtcgtacgttttgcgatcggtctcactagagcggccgcctcgaggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatccaccatgaacaagccgatcaagaatatcgtcatcgtgggcggcggtactgcgggctggatggccgcctcgtacctcgtccgggccctccaacagcaggcgaacattacgctcatcgaatctgcggcgatccctcggatcggcgtgggcgaagcgaccatcccaagtttgcagaaggtgttcttcgatttcctcgggataccggagcgggaatggatgccccaagtgaacggcgcgttcaaggccgcgatcaagttcgtgaattggagaaagtctcccgacccctcgcgcgacgatcacttctaccatttgttcggcaacgtgccgaactgcgacggcgtgccgcttacccactactggctgcgcaagcgcgaacagggcttccagcagccgatggagtacgcgtgctacccgcagcccggggcactcgacggcaagctggcaccgtgcctgtccgacggcacccgccagatgtcccacgcgtggcacttcgacgcgcacctggtggccgacttcttgaagcgctgggccgtcgagcgcggggtgaaccgcgtggtcgatgaggtggtggacgttcgcctgaacaaccgcggctacatctccaacctgctcaccaaggaggggcggacgctggaggcggacctgttcatcgactgctccggcatgcgggggctcctgatcaatcaggcgctgaaggaacccttcatcgacatgtccgactacctgctgtgcgacagcgcggtcgccagcgccgtgcccaacgacgacgcgcgcgatggggtcgagccgtacacctcctcgatcgccatgaactcgggatggacctggaagattccgatgctgggccggttcggcagcggctacgtcttctcgagccatttcacctcgcgcgaccaggccaccgccgacttcctcaaactctggggcctctcggacaatcagccgctcaaccagatcaagttccgggtcgggcgcaacaagcgggcgtgggtcaacaactgcgtctcgatcgggctgtcgtcgtgctttctggagcccctggaatcgacggggatctacttcatctacgcggcgctttaccagctcgtgaagcacttccccgacacctcgttcgacccgcggctgagcgacgctttcaacgccgagatcgtccacatgttcgacgactgccgggatttcgtccaagcgcactatttcaccacgtcgcgcgatgacacgccgttctggctcgcgaaccggcacgacctgcggctctcggacgccatcaaagagaaggttcagcgctacaaggcggggctgccgctgaccaccacgtcgttcgacgattccacgtactacgagaccttcgactacgaattcaagaatttctggttgaacggcaactactactgcatctttgccggcttgggcatgctgcccgaccggtcgctgccgctgttgcagcaccgaccggagtcgatcgagaaagccgaggcgatgttcgccagcatccggcgcgaggccgagcgtctgcgcaccagcctgccgacaaactacgactacctgcggtcgctgcgtgacggcgacgcggggctgtcgcgcggccagcgtgggccgaagctcgcagcgcaggaaagcctgtagtggaacgcaccttggaggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattgggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatccaccatgactcagaagagccccgcgaacgaacacgatagcaatcacttcgacgtaatcatcctcggctcgggcatgtccggcacccagatgggggccatcttggccaaacaacagtttcgcgtgctgatcatcgaggagtcgtcgcacccgcggttcacgatcggcgaatcgtcgatccccgagacgtctcttatgaaccgcatcatcgctgatcgctacggcattccggagctcgaccacatcacgtcgttttattcgacgcaacgttacgtcgcgtcgagcacgggcattaagcgcaacttcggcttcgtgttccacaagcccggccaggagcacgacccgaaggagttcacccagtgcgtcattcccgagctgccgtgggggccggagagccattattaccggcaagacgtcgacgcctacttgttgcaagccgccattaaatacggctgcaaggtccaccagaaaactaccgtgaccgaataccacgccgataaagacggcgtcgcggtgaccaccgcccagggcgaacggttcaccggccggtacatgatcgactgcggaggacctcgcgcgccgctcgcgaccaagttcaagctccgcgaagaaccgtgtcgcttcaagacgcactcgcgcagcctctacacgcacatgctcggggtcaagccgttcgacgacatcttcaaggtcaaggggcagcgctggcgctggcacgaggggaccttgcaccacatgttcgagggcggctggctctgggtgattccgttcaacaaccacccgcggtcgaccaacaacctggtgagcgtcggcctgcagctcgacccgcgtgtctacccgaaaaccgacatctccgcacagcaggaattcgatgagttcctcgcgcggttcccgagcatcggggctcagttccgggacgccgtgccggtgcgcgactgggtcaagaccgaccgcctgcaattctcgtcgaacgcctgcgtcggcgaccgctactgcctgatgctgcacgcgaacggcttcatcgacccgctcttctcccgggggctggaaaacaccgcggtgaccatccacgcgctcgcggcgcgcctcatcaaggcgctgcgcgacgacgacttctcccccgagcgcttcgagtacatcgagcgcctgcagcaaaagcttttggaccacaacgacgacttcgtcagctgctgctacacggcgttctcggacttccgcctatgggacgcgttccacaggctgtgggcggtcggcaccatcctcgggcagttccggctcgtgcaggcccacgcgaggttccgcgcgtcgcgcaacgagggcgacctcgatcacctcgacaacgaccctccgtatctcggatacctgtgcgcggacatggaggagtactaccagttgttcaacgacgccaaagccgaggtcgaggccgtgagtgccgggcgcaagccggccgatgaggccgcggcgcggattcacgccctcattgacgaacgagacttcgccaagccgatgttcggcttcgggtactgcatcaccggggacaagccgcagctcaacaactcgaagtacagcctgctgccggcgatgcggctgatgtactggacgcaaacccgcgcgccggcagaggtgaaaaagtacttcgactacaacccgatgttcgcgctgctcaaggcgtacatcacgacccgcatcggcctggcgctgaagaagtagccgctcgagggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattagcggccgcctcgaggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatccaccatggaacgcaccttggaccgggtaggcgtattcgcggccacccacgctgccgtggcggcctgcgatccgctgcaggcgcgcgcgctcgttctgcaactgccgggcctgaaccgtaacaaggacgtgcccggtatcgtcggcctgctgcgcgagttccttccggtgcgcggcctgccctgcggctggggtttcgtcgaagccgccgccgcgatgcgggacatcgggttcttcctggggtcgctcaagcgccacggacatgagcccgcggaggtggtgcccgggcttgagccggtgctgctcgacctggcacgcgcgaccaacctgccgccgcgcgagacgctcctgcatgtgacggtctggaaccccacggcggccgacgcgcagcgcagctacaccgggctgcccgacgaagcgcacctgctcgagagcgtgcgcatctcgatggcggccctcgaggcggccatcgcgttgaccgtcgagctgttcgatgtgtccctgcggtcgcccgagttcgcgcaaaggtgcgacgagctggaagcctatctgcagaaaatggtcgaatcgatcgtctacgcgtaccgcttcatctcgccgcaggtcttctacgatgagctgcgccccttctacgaaccgattcgagtcgggggccagagctacctcggccccggtgccgtagagatgcccctcttcgtgctggagcacgtcctctggggctcgcaatcggacgaccaaacttatcgagaattcaaagagacgtacctgccctatgtgcttcccgcgtacagggcggtctacgctcggttctccggggagccggcgctcatcgaccgcgcgctcgacgaggcgcgagcggtcggtacgcgggacgagcacgtccgggctgggctgacagccctcgagcgggtcttcaaggtcctgctgcgcttccgggcgcctcacctcaaattggcggagcgggcgtacgaagtcgggcaaagcggccccgaaatcggcagcggggggtacgcgcccagcatgctcggtgagctgctcacgctgacgtatgccgcgcggtcccgcgtccgcgccgcgctcgacgaatcctgaagcttggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattgggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatccaccatgaacgacattcaattggatcaagcgagcgtcaagaagcgtccctcgggcgcgtacgacgcaaccacgcgcctggccgcgagctggtacgtcgcgatgcgctccaacgagctcaaggacaagccgaccgagttgacgctcttcggccgtccgtgcgtggcgtggcgcggagccacggggcgggccgtggtgatggaccgccactgctcgcacctgggcgcgaacctggctgacgggcggatcaaggacgggtgcatccagtgcccgtttcaccactggcggtacgacgaacagggccagtgcgttcacatccccggccataaccaggcggtgcgccagctggagccggtgccgcgcggggcgcgtcagccgacgttggtcaccgccgagcgatacggctacgtgtgggtctggtacggctccccgctgccgctgcacccgctgcccgaaatctccgcggccgatgtcgacaacggcgactttatgcacctgcacttcgcgttcgagacgaccacggcggtcttgcggatcgtcgagaacttctacgacgcgcagcacgcaaccccggtgcacgcactcccgatctcggccttcgaactcaagctcttcgacgattggcgccagtggccggaggttgagtcgctggccctggcgggcgcgtggttcggtgccgggatcgacttcaccgtggaccggtacttcggccccctcggcatgctgtcacgcgcgctcggcctgaacatgtcgcagatgaacctgcacttcgatggctaccccggcgggtgcgtcatgaccgtcgccctggacggagacgtcaaatacaagctgctccagtgtgtgacgccggtgagcgaaggcaagaacgtcatgcacatgctcatctcgatcaagaaggtgggcggcatcctgctccgcgcgaccgacttcgtgctgttcgggctgcagaccaggcaggccgcggggtacgacgtcaaaatctggaacggaatgaagccggacggcggcggcgcgtacagcaagtacgacaagctcgtgctcaagtaccgggcgttctatcgaggctgggtcgaccgcgtcgcaagtgagcggtgaagcttggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattccggaccgcgctctagtgcgatcgcttttttaattaattttaagctttctagaggccggccgcggccgcctcgaggtaccatcaggatattcttgcttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttccttcttcttctataaaacaatacccaaagagctcttcttcttcacaattcagatttcaatttctcaaaatcttaaaaactttctctcaattctctctaccgtgatcggaaccatgacaaccttaagctgtaaagtgacctcggtagaagctatcacggataccgtatatcgtgtccgcatcgtgccagacgcggccttttcttttcgtgctggtcagtatttgatggtagtgatggatgagcgcgacaaacgtccgttctcaatggcttcgacgccggatgaaaaagggtttatcgagctgcatattggcgcttctgaaatcaacctttacgcgaaagcagtcatggaccgcatcctcaaagatcatcaaatcgtggtcgacattccccacggagaagcgtggctgcgcgatgatgaagagcgtccgatgattttgattgcgggcggcaccgggttctcttatgcccgctcgattttgctgacagcgttggcgcgtaacccaaaccgtgatatcaccatttactggggcgggcgtgaagagcagcatctgtatgatctctgcgagcttgaggcgctttcgttgaagcatcctggtctgcaagtggtgccggtggttgaacaaccggaagcgggctggcgtgggcgtactggcaccgtgttaacggcggtattgcaggatcacggtacgctggcagagcatgatatctatattgccggacgttttgagatggcgaaaattgcccgcgatctgttttgcagtgagcgtaatgcgcgggaagatcgcctgtttggcgatgcgtttgcatttatctgaggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattagcggccgcctcgaggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatctgttggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattgggtacccaattccggaccgctgctctagaggcgcgcccctagggagcttctgcagacgcgtcgacgtcatatggatccgatctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggtcgacctgcaggcatgcaagctcacgtagtgtacgtaatcgatttcgaagggccccctagtccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatc

SEQ ID NO: 35 - DNA, 20119 bp, synthetic
  misc_feature (1)..(20119): Transformation vector for plastid-targeted prnC, prnD and fre.
tggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatctatcgttctagtcgtacgttttgcgatcgcactagagcggccgcctcgaggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcagtcacacaaagagtaaagaagaacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattctgcaggaggtcgcgtcaactgcatgcaggctagcatgactcagaagagccccgcgaacgaacacgatagcaatcacttcgacgtaatcatcctcggctcgggcatgtccggcacccagatgggggccatcttggccaaacaacagtttcgcgtgctgatcatcgaggagtcgtcgcacccgcggttcacgatcggcgaatcgtcgatccccgagacgtctcttatgaaccgcatcatcgctgatcgctacggcattccggagctcgaccacatcacgtcgttttattcgacgcaacgttacgtcgcgtcgagcacgggcattaagcgcaacttcggcttcgtgttccacaagcccggccaggagcacgacccgaaggagttcacccagtgcgtcattcccgagctgccgtgggggccggagagccattattaccggcaagacgtcgacgcctacttgttgcaagccgccattaaatacggctgcaaggtccaccagaaaactaccgtgaccgaataccacaccgataaagacggcgtcgcggtgaccaccgcccagggcgaacggttcaccggccggtacatgatcgactgcggaggacctcgcgcgccgctcgcgaccaagttcaggctccgcgaagaaccgtgtcgcttcaagacgcactcgcgcagcctctacacgcacatgctcggggtcaagccgttcgacgacatcttcaaggtcaaggggcagcgctggcgctggcacgaggggaccttgcaccacatgttcgagggcggctggctctgggtgattccgttcaacaaccacccgcggtcgaccaacaacctggtgagcgtcggcctgcagctcgacccgcgtgtctacccgaaaaccgacatctccgcacagcaggaattcgatgagttcctcgcgcggttcccgagcatcggggctcagttccgggacgccgtgccggtgcgcgactgggtcaagaccgaccgcctgcaattctcgtcgaacgcctgcgtcggcgaccgctactgcctgatgctgcacgcgaacggcttcatcgacccgctcttctcccgggggctggaaaacaccgcggtgaccatccacgcgctcgcggcgcgcctcatcaaggcgctgcgcgacgacgacttctcccccgagcgcttcgagtacatcgagcgcctgcagcaaaagcttttggaccacaacgacgacttcgtcagctgctgctacacggcgttctcggacttccgcctatgggacgcgttccacaggctgtgggcggtcggcaccatcctcgggcagttccggctcgtgcaggcccacgcgaggttccgcgcgtcgcgcaacgagggcgacctcgatcacctcgacaacgaccctccgtatctcggatacctgtgcgcggacatggaggagtactaccagttgttcaacgacgccaaagccgaggtcgaggccgtgagtgccgggcgcaagccggccgatgaggccgcggcgcggattcacgccctcattgacgaacgagacttcgccaagccgatgttcggcttcgggtactgcatcaccggggacaagccgcagctcaacaactcgaagtacagcctgctgccggcgatgcggctgatgtactggacgcaaacccgcgcgccggcagaggtgaaaaagtacttcgactacaacccgatgttcgcgctgctcaaggcgtacatcacgacccgcatcggcctggcgctgaagaagtagggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattccgctcgaggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatcagtcacacaaagagtaaagaagaacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattctgcaggaggtcgcgtcaactgcatgcaggctagcatgaacgacattcaattggatcaagcgagcgtcaagaagcgtccctcgggcgcgtacgacgcaaccacgcgcctggccgcgagctggtacgtcgcgatgcgctccaacgagctcaaggacaagccgaccgagttgacgctcttcggccgtccgtgcgtggcgtggcgcggagccacggggcgggccgtggtgatggaccgccactgctcgcacctgggcgcgaacctggctgacgggcggatcaaggacgggtgcatccagtgcccgtttcaccactggcggtacgacgaacagggccagtgcgttcacatccccggccataaccaggcggtgcgccagctggagccggtgccgcgcggggcgcgtcagccgacgttggtcaccgccgagcgatacggctacgtgtgggtctggtacggctccccgctgccgctgcacccgctgcccgaaatctccgcggccgatgtcgacaacggcgactttatgcacctgcacttcgcgttcgagacgaccacggcggtcttgcggatcgtcgagaacttctacgacgcgcagcacgcaaccccggtgcacgcactcccgatctcggccttcgaactcaagctcttcgacgattggcgccagtggccggaggttgagtcgctggccctggcgggcgcgtggttcggtgccgggatcgacttcaccgtggaccggtacttcggccccctcagcatgctgtcacgcgcgctcggcctgaacatgtcgcagatgaacctgcacttcgatggctaccccggcgggtgcgtcatgaccgtcgccctggacggagacgtcaaatacaagctgctccagtgtgtgacgccggtgagcgaaggcaagaacgtcatgcacatgctcatctcgatcaagaaggtgggcggcatcctgcgccgcgcgaccgacttcgtgctgttcgggctgcagaccaggcaggccgcggggtacgacgtcaaaatctggaacggaatgaagccggacggcggcggcgcgtacagcaagtacgacaagctcgtgctcaagtaccgggcgttctatcgaggctgggtcgaccgcgtcgcaagtgagcggtgaggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattccggaccgctgctctagtgagaccgatcgcttttttaattaattttaagctttctagaggccggccgcggccgcctcgaggtaccatcaggatattcttgcttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttccttcttcttctataaaacaatacccaaagagctcttcttcttcacaattcagatttcaatttctcaaaatcttaaaaactttctctcaattctctctaccgtgatcggatcagtcacacaaagagtaaagaagaacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattctgcaggaggtcgcgtcaactgcatgcaggctagcacatgacaaccttaagctgtaaagtgacctcggtagaagctatcacggataccgtatatcgtgtccgcatcgtgccagacgcggccttttcttttcgtgctggtcagtatttgatggtagtgatggatgagcgcgacaaacgtccgttctcaatggcttcgacgccggatgaaaaagggtttatcgagctgcatattggcgcttctgaaatcaacctttacgcgaaagcagtcatggaccgcatcctcaaagatcatcaaatcgtggtcgacattccccacggagaagcgtggctgcgcgatgatgaagagcgtccgatgattttgattgcgggcggcaccgggttctcttatgcccgctcgattttgctgacagcgttggcgcgtaacccaaaccgtgatatcaccatttactggggcgggcgtgaagagcagcatctgtatgatctctgcgagcttgaggcgctttcgttgaagcatcctggtctgcaagtggtgccggtggttgaacaaccggaagcgggctggcgtgggcgtactggcaccgtgttaacggcggtattgcaggatcacggtacgctggcagagcatgatatctatattgccggacgttttgagatggcgaaaattgcccgcgatctgttttgcagtgagcgtaatgcgcgggaagatcgcctgtttggcgatgcgtttgcatttatctgagctagcggatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattagcggccgcctcgaggtaccggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagagttccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagttttgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcaggtggatctgttggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatcccccgaatttccccgatcgttcaaacatttggcaataaagtttcttaagattgaatcctgttgccggtcttgcgatgattatcatctaatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgggaattgggtacccaattccggaccgctgctctagaggcgcgcccctagggagcttctgcagacgcgtcgacgtcatatggatccgatctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggtcgacctgcaggcatgcaagctcacgtagtgtacgtaatcgatttcgaagggcccactagtccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatc